(19) United States
(12) Patent Application Publication
(10) Pub. No.: US 2021/0063381 A1
(43) Pub. Date: Mar. 4, 2021

(54) METHODS FOR SCREENING SPERM FOR ASSISTED REPRODUCTION

(71) Applicant: OHANA BIOSCIENCES, INC., Cambridge, MA (US)

(72) Inventors: Felipe A. Navarrete Solano, Medford, MA (US); Kathleen Seyb, Wakefield, MA (US); Eric Steven Furfine, Lincoln, MA (US); Robin Carl Friedman, Medford, MA (US); Joseph Brancale, New Haven, CT (US); Daniel Tien-nang Chen, Cambridge, MA (US)

(21) Appl. No.: 16/878,471

(22) Filed: May 19, 2020

Related U.S. Application Data
(60) Provisional application No. 62/895,669, filed on Sep. 4, 2019.

(51) Int. Cl. G01N 33/50; C12N 5/076; A61B 17/43

(52) U.S. Cl. CPC G01N 33/5005; A61B 17/43; C12N 5/061

(57) ABSTRACT

The present disclosure provides, inter alia, methods to determine sperm quality and suitability of a donor' sperm for a reproduction modality. The present disclosure also provides methods to identify suitable reproduction modality for a sperm sample. The methods provided herein are amenable for screening sperm samples for use in assisted fertilization. The disclosure additionally provides articles of manufacture for performing the methods provided herein. The methods provided by the disclosure, in some embodiments, entail energy depletion with subsequent staged reintroduction of different energy sources.

### CROSS-REFERENCE

This application claims the benefit of U.S. Provisional Application No. 62/895,669, filed Sep. 4, 2019, which application is incorporated herein by reference in its entirety.

### BACKGROUND

Male factor is a contributing factor for ˜50% of couples having difficulty conceiving. An important aspect of assisted reproduction is obtaining maximal function of male gametes (sperm) to help maximize fertilization. Providing suitable reproductive techniques, such as identifying the minimally invasive reproductive technique is desirable for reducing costs, increasing desired outcomes, and more quickly achieving fertilization. Accordingly, a need exists for media, compositions, methods of determining sperm quality, suitable reproductive technologies, and methods for increasing sperm function, e.g., to facilitate assisted reproduction.

### SUMMARY

In one aspect provided herein are methods of evaluating sperm quality of a sperm of a mammalian donor, comprising: (a) incubating a sample of sperm obtained from the mammalian donor under energy depletion conditions for a time suitable to generate a potentiated sperm in the sample, (b) providing or contacting the potentiated sperm in the sample from step (a) with an effective amount of a first energy source, and optionally providing or contacting the potentiated sperm with an effective amount of a second energy source, and (c) determining a sperm function in the sample from step (b) to evaluate sperm quality, wherein an increase in the sperm function relative to a suitable control indicates a higher sperm quality.

In one aspect provided herein are methods of identifying a sperm of a mammalian donor as suitable for a less invasive reproduction modality, the method comprising, (a) incubating a sample of sperm obtained from the mammalian donor under energy depletion conditions for a time suitable to generate a potentiated sperm in the sample, (b) providing or contacting the potentiated sperm in the sample from step (a) with an effective amount of a first energy source, and optionally providing or contacting the potentiated sperm with an effective amount of a second energy source, and (c) determining a sperm function in the sample from step (b), wherein an increase in the sperm function relative to a suitable control identifies the sperm of the mammalian donor as suitable for the less invasive reproduction modality.

In one aspect provided herein are methods of identifying a sperm of a mammalian donor as suitable for a reproduction modality comprising: (a) incubating a sample of sperm obtained from the mammalian donor under energy depletion conditions for a time suitable to generate a potentiated sperm in the sample, (b) providing or contacting the potentiated sperm in the sample from step (a) with an effective amount of a first energy source, and optionally providing or contacting the potentiated sperm with an effective amount of a second energy source, and (c) determining a sperm function in the sample from step (b), wherein an increase in the sperm function relative to a suitable control identifies the sperm as suitable for the reproduction modality that is a less invasive reproduction modality, and wherein a lack of increase in the sperm function relative to the suitable control identifies the sperm as suitable for the reproduction modality that is a more invasive reproduction modality. In some embodiments, the reproduction modality comprises incubating a sample of sperm obtained from the mammalian donor under energy depletion conditions for a time suitable to generate a potentiated sperm in the sample and providing the potentiated sperm in the sample from step with an effective amount of a first energy source, and optionally providing an effective amount of a second energy source.

In one aspect provided herein is a reproduction method, comprising (i) providing a sample of sperm that was: (a) obtained from the mammalian donor and was incubated under energy depletion conditions for a time suitable to generate a potentiated sperm in the sample, (b) the potentiated sperm from step (a) was contacted with an effective amount of a first energy source, and optionally an effective amount of a second energy source, and (c) a sperm function in the sample from step (b) was determined, to evaluate sperm quality, wherein an increase in the sperm function relative to a suitable control indicated a higher sperm quality, (II) wherein the method further comprises: responsive to a determination of higher sperm quality, contacting sperm from the donor with media suitable for a less-invasive reproductive modality or providing the sperm from the donor with access to an egg by a less-invasive reproductive modality; or responsive to a determination that the sperm have a lower sperm quality, contacting sperm from the donor with media suitable for a more-invasive reproductive modality or providing the sperm from the donor with access to an egg by a more-invasive reproductive modality.

In one aspect provided herein, is a reproduction method, comprising (i) providing a sample of sperm that was: (a) obtained from a mammalian donor and was incubated under energy depletion conditions for a time suitable to generate a potentiated sperm in the sample, (b) the potentiated sperm from step (a) was contacted with an effective amount of a first energy source, and optionally an effective amount of a second energy source, and (c) a sperm function in the sample from step (b) was determined, to evaluate sperm quality, wherein an increase in the sperm function relative to a suitable control indicated a higher sperm quality, (II) wherein the method further comprises: responsive to a determination of higher sperm quality, incubating a sample of sperm from the donor (e.g., sperm from the same ejaculate as the evaluated sperm, or sperm from a different ejaculate) under energy depletion conditions for a time suitable to generate a potentiated sperm in the sample, and providing an effective amount of an energy source (e.g., the first energy source), and optionally providing an effective amount of a different energy source (e.g., the second energy source); or responsive to a determination that the sperm have a lower sperm quality, incubating a sample of sperm from the donor (e.g., sperm from the same ejaculate as the evaluated sperm, or sperm from a different ejaculate) under standard capacitation conditions.

In some embodiments, the determining sperm function comprises motility as measured by computer assisted semen analysis (CASA), (optionally with classification of sperm into motility patterns by a tool such as CASAnova).

In some embodiments, the motility is determined by curvilinear velocity, percentage of hyperactivated sperm, percentage of intermediate motility sperm, or a combination thereof.

In some embodiments, the suitable control is a sperm incubated under standard capacitation conditions, non-capacitated sperm, or a combination thereof. In some embodiments, the suitable control is a sperm from the mammalian donor incubated under standard capacitation conditions. In some embodiments, suitable capacitation conditions are incubation in C-HTF media for at least 3.25 hours. In some embodiments, step (b) comprises providing or contacting the potentiated sperm with the first energy source and the second energy source concurrently or sequentially. In some embodiments, the increase in the sperm function comprises an increase in motility as measured by computer assisted semen analysis (CASA), (optionally with classification of sperm into motility patterns by a tool such as CASAnova. In some embodiments, the increase in motility comprises an increase in curvilinear velocity, an increase in percentage of hyperactivated sperm, an increase in percentage of intermediate motility sperm, or a combination thereof.

In some embodiments, the determining of step (c) comprises calculating a ratio of percent of hyperactivated sperm and intermediate motility sperm in the sperm sample relative to that in the suitable control. In some embodiments, the increase in the sperm function is indicated by the ratio of percent of hyperactivated sperm and intermediate motility sperm in the sperm (% HI) sample relative to that in the suitable control, optionally wherein the ratio is greater than 1, e.g., greater than about: 1.05, 1.1, 1.2, 1.25, 1.3, 1.35, 1.4, 1.45, 1.5, 1.55, 1.6, 1.65, 1.7, 1.75, 1.8, 1.85, 1.9, 1.95, or more, e.g., greater than about: 2, 3, 4, 5, 6, 7, 8, 9, or 10. In some embodiments, the lack of increase in the sperm function is indicated by the ratio of percent of hyperactivated sperm and intermediate motility sperm in the sperm sample relative to that in the suitable control, optionally wherein the ratio is 1 or less, e.g., less than about: 0.99, 0.95, 0.9, 0.85, 0.8, 0.75, 0.7 or less.

In some embodiments, the increase in sperm function comprises an increase in sperm capacitation as determined by a sperm-zona pellucida binding assay. In some embodiments, the increase in sperm function comprises an increase in fertilization ability of the sperm in the sample from step (b) as determined by a sperm penetration assay. In some embodiments, the increase in sperm function comprises generation of an embryo by the sperm in the sample from step (b), wherein the embryo exhibits longer viability, improved implantation, and/or ability to develop to at least a 2-cell developmental stage, blastocyst developmental stage or an offspring (i.e., a live birth) relative to an embryo generated by a suitable control.

In some embodiments, the increase in sperm function comprises an increase in amplitude of lateral head displacement. In some embodiments, the increase in sperm function comprises an increase in autophagy. In some embodiments, the reproduction modality is a less invasive reproduction modality, and wherein the less invasive reproduction modality comprises providing the sperm with access to an egg in vivo, to promote in vivo fertilization of the egg. In some embodiments, providing access in vivo comprises natural conception or artificial insemination of the sperm of the mammalian donor, e.g., by IUI, ICI, or IVI, e.g., in certain particular embodiments, by IUI.

In some embodiments, the reproduction modality is a more invasive reproduction modality, and wherein the more invasive reproduction modality comprises providing the sperm with access to an egg in vitro, to thereby promote in vitro fertilization of the egg. In some embodiments, providing access in vitro comprises incubating the sperm of the mammalian donor with the egg or injecting the sperm of the mammalian donor into the cytoplasm of the egg. In some embodiments, the sperm of the mammalian donor is incubated under energy depletion conditions for a time suitable to potentiate the sperm prior to providing access to the egg.

In some embodiments, a method described herein further comprises, responsive to a determination of lower sperm quality, contacting sperm from the donor (e.g., sperm from the same ejaculate as the evaluated sperm, or sperm from a different ejaculate) with media suitable for a more-invasive reproductive modality and/or providing the sperm from the donor with access to an egg by a more-invasive reproductive modality. In some embodiments, the determination of lower sperm quality comprises determining the % HI that is less than or equal to 75% of the % HI of the suitable control.

In some embodiments, a method described herein further comprises, responsive to a determination of higher sperm quality, contacting sperm from the donor (e.g., sperm from the same ejaculate as the evaluated sperm, or sperm from a different ejaculate) with media suitable for a less-invasive reproductive modality, and/or providing the sperm from the donor with access to an egg by a less-invasive reproductive modality. In some embodiments, the determination of higher sperm quality comprises determining the % HI that is greater than or equal to 75% of the % HI of the suitable control.

In some embodiments, the sperm of the mammalian donor is further provided with an effective amount of a first energy source, and optionally an effective amount of a second energy source prior to providing access to the egg. In some embodiments, the sample of sperm in step (a) is obtained as a pool of two or more ejaculates. In some embodiments, the mammalian donor is oligospermic or subfertile. In some embodiments, the mammalian donor is a human, non-human primate, porcine, bovine, equine, ovine, canine, feline, or murine donor. In some embodiments, the mammalian donor is human donor. In some embodiments, the sample of sperm in step (a) is recovered from a cryogenic storage.

In some embodiments, the sample of sperm in step (a) is recovered from a non-cryogenic storage. In some embodiments, the sample of sperm comprises sperm enriched from semen prior to step (a) by density gradient centrifugation, swim up, or microfluidics. In some embodiments, the method is performed at an osmolality ranging from 200-280 mOsm/kg. In some embodiments, the first energy source is a glycolytic energy source, or a gluconeogenesis substrate. In some embodiments, the second energy source is a glycolytic energy source or a gluconeogenesis substrate, and wherein the second energy source is one not selected as the first energy source. In some embodiments, the glycolytic energy source is glucose and the gluconeogenesis substrate is pyruvate.

In some embodiments, the incubating under energy depletion conditions comprises incubating in a medium comprising glucose concentration of less than about: 0.5, 0.4, 0.3, 0.2, 0.1, 0.09, 0.08, 0.07, 0.06, 0.05, 0.04, 0.03 mM, or less. In some embodiments, the incubating under energy depletion conditions comprises incubating in a medium comprising pyruvate concentration of less than about: 0.15, 0.10, 0.09, 0.08, 0.07, 0.06, 0.05, 0.04, 0.03, 0.02, 0.01, 0.005, 0.003, 0.002 mM, or less.

In some embodiments, the incubating under energy depletion conditions is for at least about: 10, 20, 30, 40, 45, 50, 55, 60, 90, 120, 150, or 180 minutes. In some embodiments, the time between providing the effective amount of the first energy source and the effective amount of the second energy is at least about 1 min to 60 minutes. In some embodiments, the glycolytic energy source is glucose, wherein the glucose is provided at the effective amount of between about: 0.6 mM-10 mM. In some embodiments, the gluconeogenesis substrate is pyruvate, wherein the pyruvate is provided at the effective amount of between about: 0.15 mM-0.66 mM.

Provided herein are kits for identifying a sperm of a mammalian donor as suitable for a reproduction modality comprising, (a) a first container comprising a sperm potentiating solution, that upon contact with a sample of sperm from the mammalian donor induces energy depletion conditions, (b) a second container comprising a solution comprising an effective amount of at least a first energy source, such as a glycolytic energy source, or a gluconeogenesis substrate, and (c) optionally a third container comprising a solution comprising an effective amount of a second energy source, such as a glycolytic energy source, or a gluconeogenesis substrate, and wherein the second energy source is one that is not selected as the first energy source. In some embodiments, the kit further comprises a microscope slide. In some embodiments, the kit further comprises instructions for identifying a sperm of a mammalian donor as suitable for a reproduction modality comprising.

In some embodiments, the kit further comprises a collection container for collecting a sperm sample from the mammalian donor. In some embodiments, the kit further comprises a written instruction sheet. In some embodiments, the first container, the second container, and the optional the third container is a bottle, a vial, a syringe, or a test tube. In some embodiments, the first container, the second container, and the optional third container is a multi-use container. In some embodiments, the glycolytic energy source is glucose and the gluconeogenesis substrate is pyruvate.

In some embodiments, the sperm potentiating solution comprises glucose at a concentration of less than about: 0.5, 0.4, 0.3, 0.2, 0.1, 0.09, 0.08, 0.07, 0.06, 0.05, 0.04, 0.03 mM or less. In some embodiments, the sperm potentiating solution comprises pyruvate at a concentration of less than about: 0.15, 0.10, 0.09, 0.08, 0.07, 0.06, 0.05, 0.04, 0.03, 0.02, 0.01, 0.005, 0.003, 0.002 mM, or less. In some embodiments, the glycolytic energy source is glucose, wherein the glucose is provided at the effective amount of between about 0.6 mM-10 mM. In some embodiments, the gluconeogenesis substrate is pyruvate, wherein the pyruvate is provided at the effective amount of between about: 0.15 mM-0.66 mM.

In some embodiments, the kit further comprises a sperm isolating matrix. In some embodiments, the sperm isolating matrix is silanized silica, optionally wherein the silanized silica is in media substantially free of any glycolytic energy source or gluconeogenesis substrate.

Provided herein is a reproduction method, comprising providing a sperm of a mammalian donor with access to an egg by a reproduction modality, wherein the reproduction modality was previously identified by a method comprising, (a) incubating a sample of sperm obtained from the mammalian donor under energy depletion conditions for a time suitable to generate a potentiated sperm in the sample, (b) providing the potentiated sperm in the sample from step (a) with an effective amount of a first energy source, and optionally providing an effective amount of a second energy source, and (c) determining a sperm function in the sample from step (b) to identify the reproduction modality, wherein an increase in the sperm function relative to a suitable control identifies the reproduction modality as a less invasive reproduction modality, and wherein a lack of increase in the sperm function relative to the suitable control identifies the reproduction modality as a more invasive reproduction modality.

In some embodiments, the sperm of the mammalian donor and/or the sample of sperm is provided as a pool of two or more ejaculates. In some embodiments, the sperm of the mammalian donor and/or the sample of the sperm is recovered from a cryogenic storage. In some embodiments, the sperm of the mammalian donor and/or the sample of sperm is recovered from a non-cryogenic storage. In some embodiments, the sperm of the mammalian donor and/or the sample of sperm has been enriched from semen by density gradient centrifugation, swim up, or microfluidics. In some embodiments, prior to providing the sperm with access to the egg, the reproduction method comprises the step of incubating the sperm under energy depletion conditions for a time suitable to potentiate the sperm.

In some embodiments, the method further comprises providing the sperm with an effective amount of a first energy source, and optionally providing an effective amount of a second energy source prior to providing the sperm with access to the egg. In some embodiments, the less invasive reproduction modality comprises providing the sperm with access to the egg in vivo, to promote in vivo fertilization of the egg. In some embodiments, providing access in vivo comprises natural conception or artificial insemination of the sperm of the mammalian donor.

In some embodiments, the more invasive reproduction modality comprises providing access to the egg in vitro, to promote in vitro fertilization of the egg. In some embodiments, the providing access in vitro comprises incubating the sperm of the mammalian donor with the egg or injecting the sperm of the mammalian donor into the cytoplasm of the egg.

Provided herein is a reproduction method, comprising providing a sperm of a mammalian donor with access to an egg by a reproduction modality, wherein a sperm sample from the mammalian donor was previously evaluated by the method disclosed above.

### ENUMERATED EMBODIMENTS

1. A method of evaluating sperm quality of a sperm of a mammalian donor, comprising:

(a) incubating a sample of sperm obtained from the mammalian donor under energy depletion conditions for a time suitable to generate a potentiated sperm in the sample;

(b) contacting the potentiated sperm in the sample from step (a) with an effective amount of a first energy source, and optionally contacting the potentiated sperm with an effective amount of a second energy source; and

(c) determining a sperm function in the sample from step (b) to evaluate sperm quality,

wherein an increase in the sperm function relative to a suitable control indicates a higher sperm quality.

2. A method of preparing sperm of a mammalian donor for evaluation of sperm quality, comprising:

(a) incubating a sample of sperm obtained from the mammalian donor under energy depletion conditions for a time suitable to generate a potentiated sperm in the sample;

(b) contacting the potentiated sperm in the sample from step (a) with an effective amount of a first energy source, and a second energy source in a serial manner; and

(c) placing sperm from the sample from step (b) onto a microscope slide (e.g., a pre-warmed microscope slide).

3. A method of evaluating sperm quality of a sperm of a mammalian donor, comprising:

(a) providing a sample of sperm that was obtained from the mammalian donor and was incubated under energy depletion conditions for a time suitable to generate a potentiated sperm in the sample (e.g., wherein the providing comprises obtaining the sample from a third party that performed the incubation under energy depletion conditions, or wherein the providing comprises performing the incubation under energy depletion conditions);

(b) contacting the potentiated sperm in the sample from step (a) with an effective amount of a first energy source, and optionally contacting the potentiated sperm with an effective amount of a second energy source; and

(c) determining a sperm function in the sample from step (b) to evaluate sperm quality, wherein an increase in the sperm function relative to a suitable control indicates a higher sperm quality.

4. A method of evaluating sperm quality of a sperm of a mammalian donor, comprising:

providing a sample of sperm that was:
(a) obtained from the mammalian donor and was incubated under energy depletion conditions for a time suitable to generate a potentiated sperm in the sample (e.g., wherein the providing comprises obtaining the sample from a third party that performed the incubation under energy depletion conditions, or wherein the providing comprises performing the incubation under energy depletion conditions); and
(b) the potentiated sperm from step (a) was provided or contacted with an effective amount of a first energy source, and optionally an effective amount of a second energy source (e.g., wherein the provision of or contacting with the first and/or second energy source was performed by the third party of step (a));

wherein the method further comprises:
(c) determining a sperm function in the sample from step (b) to evaluate sperm quality, wherein an increase in the sperm function relative to a suitable control indicates a higher sperm quality.

5. A method of identifying a sperm of a mammalian donor as suitable for a reproduction modality comprising:

(a) incubating a sample of sperm obtained from the mammalian donor under energy depletion conditions for a time suitable to generate a potentiated sperm in the sample;

(b) contacting the potentiated sperm in the sample from step (a) with an effective amount of a first energy source, and optionally contacting the potentiated sperm with an effective amount of a second energy source; and

(c) determining a sperm function in the sample from step (b), wherein an increase in the sperm function relative to a suitable control identifies the sperm as suitable for the reproduction modality that is a less invasive reproduction modality, and wherein a lack of increase in the sperm function relative to the suitable control identifies the sperm as suitable for the reproduction modality that is a more invasive reproduction modality. 6. A reproduction method, comprising:

(I) providing a sample of sperm that was:
(a) obtained from the mammalian donor and was incubated under energy depletion conditions for a time suitable to generate a potentiated sperm in the sample;
(b) the potentiated sperm from step (a) was contacted with an effective amount of a first energy source, and optionally an effective amount of a second energy source; and
(c) a sperm function in the sample from step (b) was determined, to evaluate sperm quality, wherein an increase in the sperm function relative to a suitable control indicated a higher sperm quality;

(II) wherein the method further comprises:
responsive to a determination of higher sperm quality, contacting sperm from the donor with media suitable for a less-invasive reproductive modality or providing the sperm from the donor with access to an egg by a less-invasive reproductive modality;
responsive to a determination that the sperm have a lower sperm quality, contacting sperm from the donor with media suitable for a more-invasive reproductive modality or providing the sperm from the donor with access to an egg by a more-invasive reproductive modality.

7. A reproduction method, comprising:

(I) providing a sample of sperm that was:
(a) obtained from a mammalian donor and was incubated under energy depletion conditions for a time suitable to generate a potentiated sperm in the sample;
(b) the potentiated sperm from step (a) was contacted with an effective amount of a first energy source, and optionally an effective amount of a second energy source; and
(c) a sperm function in the sample from step (b) was determined, to evaluate sperm quality, wherein an increase in the sperm function relative to a suitable control indicated a higher sperm quality;

(II) wherein the method further comprises:
responsive to a determination of higher sperm quality, incubating a sample of sperm from the donor (e.g., sperm from the same ejaculate as the evaluated sperm, or sperm from a different ejaculate) under energy depletion conditions for a time suitable to generate a potentiated sperm in the sample, and providing an effective amount of an energy source (e.g., the first energy source), and optionally providing an effective amount of a different energy source (e.g., the second energy source);

responsive to a determination that the sperm have a lower sperm quality, incubating a sample of sperm from the donor (e.g., sperm from the same ejaculate as the evaluated sperm, or sperm from a different ejaculate) under standard capacitation conditions.

8. A method of identifying sperm quality, the method comprising:

(I) acquiring knowledge by (a) incubating a sample of sperm obtained from a mammalian donor under energy depletion conditions for a time suitable to generate a potentiated sperm in the sample; (b) contacting the potentiated sperm in the sample from step (a) with an effective amount of a first energy source, and optionally contacting the potentiated sperm with an effective amount of a second energy source; and (c) determining a sperm function in the sample from step (b) to evaluate sperm quality, wherein an increase in the sperm function relative to a suitable control indicates a higher sperm quality, and

(II) providing a report of sperm quality to another party, e.g., an insurance company or third party payor.

9. The method of embodiment 8, wherein the report comprises one or more of:
(a) information on the evaluation of sperm quality;
(b) information on a suggested reproductive modality for the donor;
(c) information on the likely effectiveness of a reproductive modality or the advisability of performing a reproductive modality; or
(d) information or a recommendation on, the use of a reproductive modality.

10. The method of embodiments 8 or 9, wherein the report is in electronic, web-based, or paper form.

11. The method of any of embodiments 8-10, wherein the third party is the donor, a physician, a hospital, a clinic, a third-party payor, an insurance company, an office, or a laboratory.

12. A method of making a decision to fund a procedure based on sperm quality, the method comprising:

(I) acquiring knowledge from a third party (e.g., a a laboratory or clinic) about sperm quality, wherein the third party: (a) incubated a sample of sperm obtained from a mammalian donor under energy depletion conditions for a time suitable to generate a potentiated sperm in the sample; (b) contacted the potentiated sperm in the sample from step (a) with an effective amount of a first energy source, and optionally contacted the potentiated sperm with an effective amount of a second energy source; and (c) determined a sperm function in the sample from step (b) to evaluate sperm quality, wherein an increase in the sperm function relative to a suitable control indicates a higher sperm quality, and

(II) if the sperm is higher quality, then funding the procedure, and if the sperm is not higher quality, then not funding the procedure.

13. The method of embodiment 12, wherein the acquiring knowledge of step (I) comprises obtaining a report of the sperm quality from the third party.

14. The method of embodiment 12 or 13, wherein the procedure comprises incubating a sample of sperm obtained from the donor under energy depletion conditions for a time suitable to generate a potentiated sperm in the sample and providing the potentiated sperm in the sample from step with an effective amount of an energy source (e.g., the first energy source), and optionally providing an effective amount of a different energy source (e.g., a second energy source).

15. The method of embodiment 2, wherein the sperm from step (b) are resuspended, e.g., by flicking a container comprising the sperm.

16. The method of any of embodiments 1-8 or 12, wherein the suitable control is a sperm incubated under standard capacitation conditions, non-capacitated sperm, or a combination thereof.

17. The method of any of embodiments 1-8 or 12, wherein the suitable control is a sperm from the mammalian donor incubated under standard capacitation conditions.

18. The method of embodiment 16 or 17, wherein the standard capacitation conditions are incubation at about 34° C. to about 37° C. (e.g., 37° C.), in C-HTF media for about 3 hours or for the length of time that the sperm being evaluated are undergoing steps (a) and (b).

19. The method of any of the preceding embodiments, wherein the method is performed at an osmolality ranging from 200-280 mOsm/kg.

20. The method of any of the preceding embodiments, wherein the method is performed at about 34° C. to about 37° C. (e.g., 37° C.).

21. The method of any of embodiments 1 or 3-20, wherein step (b) comprises contacting the potentiated sperm with the first energy source and the second energy source concurrently.

22. The method of any of embodiments 1-20, wherein step (b) comprises contacting the potentiated sperm with the first energy source and the second energy source sequentially.

23. The method of any of the preceding embodiments, wherein the first energy source is a glycolytic energy source, or a gluconeogenesis substrate.

24. The method of any of the preceding embodiments, wherein the second energy source is a glycolytic energy source or a gluconeogenesis substrate, and wherein the second energy source is one not selected as the first energy source.

25. The method of any of embodiments 1-24, wherein the first energy source comprises glucose and the second energy source comprises pyruvate.

26. The method of any of embodiments 1-24, wherein the first energy source comprises pyruvate and the second energy source comprises glucose.

27. The method of any of the preceding embodiments, wherein (b) comprises incubating the sperm cells with the first energy source for 1 hour.

28. The method of any of the preceding embodiments, wherein (b) comprises incubating the sperm cells with the second energy source for 1 hour.

29. The method of any of the preceding embodiments, wherein the time suitable to generate a potentiated sperm is about 1 hour.

30. The method of any of the preceding embodiments, wherein the mammalian donor is oligospermic or subfertile.

31. The method of any of the preceding embodiments, wherein the mammalian donor is a human.

32. The method of any of embodiments 1 or 3-31, wherein the determining sperm function of step (c) comprises determining motility by computer assisted semen analysis (CASA), (optionally with classification of sperm into motility patterns by a tool such as CASAnova), sperm capacitation by a sperm-zona pellucida binding assay, ability to fertilize an egg by a sperm penetration assay, autophagy, generation of an embryo, or a combination thereof.

33. The method of embodiment 32, wherein the determining sperm function of step (c) comprises classification of the sperm motility, e.g., weak, slow, progressive, intermediate, or hyperactivated, e.g., by CASAnova.

34. The method of embodiment 32 or 33, wherein the determining sperm function of step (c) comprises classification of the sperm motility, e.g., weak, slow, progressive, intermediate, or hyperactivated, according to the criteria in Goodson et al. 2017, Biol. Reprod. 97:698-708.

35. The method of any of embodiments 32-34 wherein the motility is determined by average path velocity (VAP), curvilinear velocity (VCL), amplitude of lateral head displacement (ALH), linearity of forward progression (LIN), or a combination thereof.

36. The method of embodiment 32-35 wherein the motility is determined by percentage of hyperactivated sperm, percentage of intermediate motility sperm, or a combination thereof.

37. The method of any of embodiments 1 or 3-36, wherein the increase in sperm function comprises an increase curvilinear velocity, amplitude of lateral head displacement, percentage of hyperactivated sperm, percentage of intermediate motility sperm, autophagy, or a combination thereof.

38. The method of any of embodiments 1 or 3-36, wherein the determining sperm function of step (c) comprises calculating a ratio of percent of hyperactivated sperm and intermediate motility sperm in the sperm sample relative to that in the suitable control.

39. The method of any of embodiments 1 or 3-38, wherein the sperm having a higher sperm quality have a % HI (percentage of total cells classified as either Hyperactivated or Intermediate) that is greater than or equal to 125% of the % HI of the suitable control (e.g., wherein the suitable control is a sperm from the mammalian donor incubated under standard capacitation conditions).

40. The method of any of the preceding embodiments, wherein the sperm from step (b) have a % HI (percentage of total cells classified as either Hyperactivated or Intermediate) that is greater than or equal to 100%, 105%, 110%, 115%, 120%, 125%, 150%, 200%, 250%, or 300% of the % HI of the suitable control.

41. The method of any of the preceding embodiments, wherein the sperm from step (b) have a % HI (percentage of total cells classified as either Hyperactivated or Intermediate) that is less than or equal to 100%, 75%, 50%, or 25% of the % HI of the suitable control.

42. The method of any of embodiments 1 or 3-41, wherein the increase in sperm function comprises generation of an embryo by the sperm in the sample from step (b), wherein the embryo exhibits longer viability, improved implantation, and/or ability to develop to at least a 2-cell developmental stage, blastocyst developmental stage or an offspring (i.e., a live birth) relative to an embryo generated by a suitable control.

43. The method of any of the preceding embodiments, which further comprises providing sperm from the donor (e.g., sperm from the same ejaculate as the evaluated sperm, or sperm from a different ejaculate) with access to an egg by a reproduction modality.

44. The method of any of the preceding embodiments, which further comprises:

(a) incubating sperm from the donor (e.g., sperm from the same ejaculate as the evaluated sperm, or sperm from a different ejaculate) under energy depletion conditions for a time suitable to generate a potentiated sperm in the sample; and

(b) contacting the potentiated sperm from step (a) with an effective amount of a first energy source, and optionally contacting the potentiated sperm with an effective amount of a second energy source.

45. The method of any of the preceding embodiments, which further comprises contacting sperm from the donor (e.g., sperm from the same ejaculate as the evaluated sperm, or sperm from a different ejaculate, and optionally sperm that has undergone steps (a) and (b) above) with a fertilization buffer.

46. The method of any of the preceding embodiments, which further comprises providing the sperm from the donor (e.g., sperm in fertilization buffer) with access to an egg in a drop of media suitable for a more invasive reproductive modality (e.g., 50-150 μL, e.g., 100 μL, of media) and optionally contacting the drop with oil.

47. The method of any of the preceding embodiments, which further comprises:

(a) incubating sperm from the donor (e.g., sperm from the same ejaculate as the evaluated sperm, or sperm from a different ejaculate) under energy depletion conditions for a time suitable to generate a potentiated sperm in the sample;

(b) contacting the potentiated sperm from step (a) with an effective amount of a first energy source, and optionally contacting the potentiated sperm with an effective amount of a second energy source;

(c) contacting the sperm from step (b) with a fertilization buffer; and

(d) providing the sperm from step (c) access to an egg in a drop of media suitable for a more invasive reproductive modality (e.g., 50-150 μL, e.g., 100 μL, of media) and contacting the drop with oil.

48. The method of embodiment 47, wherein step (d) comprises concurrent or sequential addition of the sperm from step (c) and the egg into the drop of media.

49. The method of embodiment 47 or 48, wherein step (d) comprises addition of the sperm from step (c) to the drop of media, followed by the addition of the egg to the drop of media.

50. The method of embodiment 47 or 48, wherein step (d) comprises addition of the egg to the drop of media, followed by the addition of the sperm from step (c) to the drop of media.

51. The method of any of the embodiments 1-43, which further comprises:

(a) incubating sperm from the donor (e.g., sperm from the same ejaculate as the evaluated sperm, or sperm from a different ejaculate) under energy depletion conditions for a time suitable to generate a potentiated sperm in the sample;

(b) contacting the potentiated sperm from step (a) with an effective amount of an energy source; and

(c) providing access to an egg by a less invasive reproductive modality, e.g., in vivo.

52. The method of 51, wherein the energy source comprises a gluconeogenesis substrate, e.g., pyruvate.

53. The method of any of the preceding embodiments, which further comprises contacting sperm from the donor (e.g., sperm from the same ejaculate as the evaluated sperm, or sperm from a different ejaculate) with media suitable for a more-invasive reproductive modality (e.g., ART, e.g., GIFT, IVF (e.g., ICSI BT, and/or ZIFT)).

54. The method of any of the preceding embodiments, which further comprises, responsive to a determination of lower sperm quality, contacting sperm from the donor (e.g., sperm from the same ejaculate as the evaluated sperm, or sperm from a different ejaculate) with media suitable for a more-invasive reproductive modality and/or providing the sperm from the donor with access to an egg by a more-invasive reproductive modality.

55. The method of any of the preceding embodiments, which further comprises, responsive to a determination of lower sperm quality, obtaining a sperm sample from a second donor, e.g., for use in a reproductive modality.

56. The method of embodiment 54 or 55, wherein the determination of lower sperm quality comprises determining that the % HI is less than or equal to 75% of the % HI of the suitable control.

57. The method of any of the preceding embodiments, which further comprises contacting sperm from the donor (e.g., sperm from the same ejaculate as the evaluated sperm, or sperm from a different ejaculate) with media suitable for a less-invasive reproductive modality (e.g., IUI, ICI, or IVI).

58. The method of any of embodiments 1-53, which further comprises, responsive to a determination of higher sperm quality, contacting sperm from the donor (e.g., sperm from the same ejaculate as the evaluated sperm, or sperm from a different ejaculate) with media suitable for a less-invasive reproductive modality, and/or providing the sperm from the donor with access to an egg by a less-invasive reproductive modality.

59. The method of embodiment 58, wherein the determination of higher sperm quality comprises determining the % HI that is greater than or equal to 125% of the % HI of the suitable control.

60. A reproduction method, comprising providing a sperm of a mammalian donor with access to an egg by a reproduction modality, wherein a sperm sample from the mammalian donor was previously evaluated by the method of any of embodiments 1-8.

61. The method of embodiment 5-7, 43, 57-58, or 60, wherein the reproduction modality is a less invasive reproduction modality, and wherein the less invasive reproduction modality comprises providing the sperm with access to an egg in vivo, to promote in vivo fertilization of the egg, by IUI, ICI, or IVI.

62. The method of embodiment 5-7, 43, 53-55, or 60, wherein the reproduction modality is a more invasive reproduction modality, and wherein the more invasive reproduction modality comprises providing the sperm with access to an egg in vitro, to thereby promote in vitro fertilization of the egg, wherein the providing access in vitro comprises incubating the sperm of the mammalian donor with the egg or injecting the sperm of the mammalian donor into the cytoplasm of the egg.

63. The method of embodiment 5-7, 43, 53-55, 57-58, or 60-62, wherein the reproduction modality comprises incubating a sample of sperm obtained from the mammalian donor under energy depletion conditions for a time suitable to generate a potentiated sperm in the sample and providing the potentiated sperm in the sample from step with an effective amount of a first energy source, and optionally providing an effective amount of a second energy source.

### BRIEF DESCRIPTION OF THE DRAWINGS

FIG. 1 is a bar graph of the Mean Percent HI for N=123 donors subjected to SC, SR, nG, nP treatments. Error bars indicate 95% confidence interval for the mean.

FIG. 2 is a bar graph of the Mean Fold Change in Percent HI of SR, nP, nG treated samples relative to SC treated samples for N=123 donors. Error bars indicate 95% confidence interval for the mean.

FIG. 3 is a scatterplot of Log 2 Fold Change in Percent HI of SR treatment vs Percent HI of SC treatment. Circles, triangles, and square symbols indicate unknown, responders, and non-responder classes. NB: In this plot FC HI=1, no enhancement in Percent HI, corresponds to a value of Log 2 (FC HI)=0.

### DETAILED DESCRIPTION OF THE INVENTION

Male factor is a contributing factor for ˜50% of couples having difficulty conceiving. Low sperm count is a recognized factor in male infertility. The World Health Organization defines low sperm count (oligospermia) as less than 15 million sperm per milliliter (Cooper et al., Human Reproduction Update, 16(3), 231-245, 2009). Other factors contributing to male infertility or subfertility include low motility or abnormal morphology. An important aspect of assisted reproduction is obtaining maximal function of male gametes (sperm) to help maximize fertilization. Before fertilization, sperm must go through a series of changes to be able to fertilize the egg, a process called sperm capacitation. In vitro capacitation media includes three components (albumin, calcium and bicarbonate) and initiate sperm capacitation. Sperm initially swim progressively with an almost symmetrical flagellar movement. After different periods of time, which depend on the species, the straight sperm movement is replaced by an in-place helical movement known as “hyperactivation”. While methods for activating sperm exist, they fail to achieve maximal sperm activation and therefore do not adequately address the impact of male factor in infertility. Accordingly, a need exists for media, compositions, and methods for increasing sperm function, e.g., to facilitate assisted reproduction.

The present disclosure provides, inter alia, methods for evaluating sperm quality, identifying a suitable reproduction modality for a sperm sample, and articles of manufacture, e.g., useful for performing methods provided by the disclosure. The disclosure is based, at least in part, on Applicant's surprising discovery that the presence, absence, or degree of increased sperm function in response to reintroduction of one or more energy sources after a period of starvation is a useful predictor of sperm quality and/or success of different reproductive techniques, such as certain assisted reproductive technologies.

### Definitions

To facilitate an understanding of the present disclosure, a number of terms and phrases are defined below.

The terms “increased”, ‘increase”, “increasing” or “enhance” or “promote” are all used herein to generally mean an increase; for the avoidance of doubt, the terms “increased”, “increase”, or “enhance”, mean an increase of at least 5%, e.g., at least 10% as compared to a suitable control, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a suitable control, or at least about a 2-fold, or at least about a 3-fold, or at least about a 4-fold, or at least about a 5-fold or at least about a 10-fold increase, or any increase between 2-fold and 10-fold or greater as compared to a suitable control. The increase can be, for example, at least 10%, at least 20%, at least 30%, at least 40% or more, and is preferably to a level accepted as within the range of normal sperm from a mammalian male subject without a given disease (e.g., male infertility, due to abnormal sperm function or oligospermia).

The terms, “decrease”, “reduce”, “reduction”, “lower” or “lowering,” or “inhibit” are all used herein generally to mean a decrease. For example, “decrease”, “reduce”, “reduction”, or “inhibit” means a decrease by at least 5%, e.g., 10% as compared to a suitable control, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a suitable control), or any decrease between 10-100% as compared to a suitable control. The decrease can be, for example, at least 10%, at least 20%, at least 30%, at least 40% or more, than the range of normal for an individual without a given disease.

“Acquire” or “acquiring” as the terms are used herein, refer to obtaining possession of a physical entity, or a value, e.g., a numerical value, by “directly acquiring” or “indirectly acquiring” the physical entity or value. “Directly acquiring” means performing a process (e.g., performing a synthetic or analytical method) to obtain the physical entity or value. “Indirectly acquiring” refers to receiving the physical entity or value from another party or source (e.g., a third party laboratory that directly acquired the physical entity or value). Directly acquiring a physical entity includes performing a process that includes a physical change in a physical substance, e.g., a starting material. Exemplary changes include making a physical entity from two or more starting materials, shearing or fragmenting a substance, separating or purifying a substance, combining two or more separate entities into a mixture, performing a chemical reaction that includes breaking or forming a covalent or non-covalent bond. Directly acquiring a value includes performing a process that includes a physical change in a sample or another substance, e.g., performing an analytical process which includes a physical change in a substance, e.g., a sample, analyte, or reagent (sometimes referred to herein as “physical analysis”), performing an analytical method, e.g., a method which includes one or more of the following: separating or purifying a substance, e.g., an analyte, or a fragment or other derivative thereof, from another substance; combining an analyte, or fragment or other derivative thereof, with another substance, e.g., a buffer, solvent, or reactant; or changing the structure of an analyte, or a fragment or other derivative thereof, e.g., by breaking or forming a covalent or non-covalent bond, between a first and a second atom of the analyte; or by changing the structure of a reagent, or a fragment or other derivative thereof, e.g., by breaking or forming a covalent or non-covalent bond, between a first and a second atom of the reagent.

As used herein, the term “effective amount” means the total amount of the active component(s) of a first energy source or a second energy source that is sufficient to cause a change on a detectable function of the mammalian sperm (e.g., sperm motility, curvilinear velocity, amplitude of lateral head displacement, autophagy, sperm capacitation, percentage of hyperactivated sperm, percentage of intermediate motility sperm and percentage of hyperactivated sperm and intermediate motility sperm, ability to fertilize an egg, and generation of an embryo). When applied to an individual energy source, administered alone, the term refers to that energy source alone. When applied to a combination, the term refers to combined amounts of the first energy source and the second energy source that result in the effect, whether administered in combination, serially or simultaneously.

The term “an effective amount” includes within its meaning a sufficient amount of an energy source (e.g., a gluconeogenesis substrate or glycolytic energy source) to provide the desired effect. As it relates to the present disclosure, the desired effect can be increase in one or more sperm function or increase in fertilization. The exact amount required will vary depending on factors such as the mammalian sperm species being treated, the age and general condition of the male subject from whom the mammalian sperm is obtained, for example if the sperm is obtained from a sub-fertile mammalian subject. Thus, it is not possible to specify an exact “effective amount”. However, for any given case, an appropriate “effective amount” may be determined by one of ordinary skill in the art using only routine experimentation.

The term “energy depletion” refers to suppressing or restricting the energetic output of a cell whether by depletion, reduction (below an effective amount), or removal of such energy sources or inhibition of enzymatic or import machinery.

The term “standard capacitation conditions” as used herein refers to incubating sperm in standard capacitation media such as, human tubal fluid (“HTF”) medium or modified HTF medium and not in energy depletion conditions.

The term “potentiate” or “potentiating” sperm means to condition sperm such that, upon a suitable induction, e.g., removing or reversing the energy depletion and, e.g., incubating the sperm in capacitation conditions or staged energy reintroduction, the sperm rapidly recover motility, such as one or more of: an increased proportion of hyperactivated, intermediate, or progressive motility sperm (or an increased proportion of a combination of two (such as hyperactivated and intermediate) or all three), and/or increased curvilinear velocities.

The term “spermatozoon” refers to a live reproductive cell from a male mammal. The term “spermatozoa” refers to a plurality of live male reproductive cells. Unless required otherwise by context, the plural and singular forms are interchangeable. The term “sperm” is used as an abbreviation and refers to at least one spermatozoon.

As used herein, the term “ability to fertilize an egg” refers to ability of a sperm (e.g., mammalian sperm) to penetrate an unfertilized egg (ovum) resulting in combination of their genetic material resulting in the formation of a zygote. As it relates to the present disclosure, the “ability to fertilize” an egg can be ability to fertilize in vitro and/or in vivo. In some embodiments, ability to fertilize in vitro comprises fertilization by intracytoplasmic sperm injection (ICSI).

The term “embryo” is used herein to refer both to the zygote that is formed upon fertilization of an unfertilized egg by a mammalian sperm, to form a diploid totipotent cell, e.g. a fertilized egg and to the embryo that undergoes subsequent cell divisions to develop to 2-cell stage or greater (e.g., 4-cell stage, 16-cell stage, 32-cell stage, the blastocyst stage (with differentiated trophectoderm and inner cell mass) or development into an offspring).

As used herein, the term “ability to develop” refers to the ability or capacity of an embryo to grow or develop. The terms may refer to the ability or capacity of an embryo to reach at least the 2-cell developmental stage, the blastocyst developmental stage, implant into the uterus, to develop to a full offspring, or be born live. The term “offspring” as used herein refers to a progeny of a parent, wherein the progeny is an unborn fetus or a newborn.

The term “blastocyst” refers to an embryo, five or six days after fertilization, having an inner cell mass, an outer cell layer called the trophectoderm, and a fluid-filled blastocele cavity containing the inner cell mass from which the whole of the embryo is derived. The trophectoderm is the precursor to the placenta. The blastocyst is surrounded by the zona pellucida which is subsequently shed when the blastocyst “hatches.” The zona pellucida, composed of a glycoprotein coat, surrounds the oocyte from the one-cell stage to the blastocyst stage of development. Prior to embryo attachment and implantation, the zona pellucida is shed from the embryo by a number of mechanisms including proteolytic degradation. The zona pellucida functions initially to prevent entry into the oocyte by more than one sperm, then later to prevent premature adhesion of the embryo before its arrival into the uterus.

The terms “activity” and/or “function” refers to physiological processes such as, for example, sperm motility, sperm tropism (namely, the tendency of sperm to move towards or away from certain stimuli), and ability to fertilize an egg. The terms “activity” and/or “function” can further include processes which occur prior to, during fertilization and/or interaction with the egg (or membranes/layers thereof)—such processes may include, for example sperm capacitation and acrosomal activity, and/or processes after fertilization of egg, for example, formation of an embryo.

The term “sperm quality” refers to the ability of sperm to fertilize an egg or oocyte leading to generation of an embryo that is able to give rise to pregnancy or other measures of sperm quality as described, below.

The term “higher sperm quality” refers to sperm or spermatozoa comprising an increase in sperm function leading to increase in fertility rate. This increase in sperm function can be relative to a suitable control.

The term “suitable for a reproduction modality” refers to a reproduction modality having the greatest or highest probability for success resulting in fertilization of an egg leading to embryo generation and pregnancy. In some embodiments, the reproduction modality is a “less invasive reproduction modality”, In some embodiments, the reproduction modality is a “more invasive reproduction modality”.

As used herein, the term “less invasive reproduction modality” refers to a reproduction method comprising providing a sperm of a mammalian donor with access to an egg in vivo, for in vivo fertilization of the egg. In some embodiments, a sperm is provided access to an egg by natural conception. In some embodiments, a sperm is provided access to an egg by artificial insemination. The artificial insemination can be intrauterine insemination (IUI) or intracervical insemination.

As used herein, the term “more invasive reproduction modality” refers to a reproduction method comprising providing a sperm of a mammalian donor with access to an egg in vitro, for in vitro fertilization of the egg. The more invasive reproduction modality can entail transfer of the fertilized egg into the uterus of a female subject. In some embodiments, a sperm is provided access to an egg by incubating the sperm with the egg under conditions suitable for in vitro fertilization of the egg (e.g., IVF). In some embodiments, a sperm is provided access to an egg by injecting the sperm into cytoplasm of an egg (e.g., ICSI).

The term “assisted reproductive technologies” or “ART” or “assisted fertilization” has its general meaning in the art and refers to methods used to achieve pregnancy by artificial or partially artificial means. Assisted reproductive technologies include but are not limited to classical in vitro fertilization (IVF), intracytoplasmic sperm injection (ICSI), intrauterine insemination (IUI), and intracervical insemination.

The term “intrauterine insemination” or “IUI” refers to intrauterine injection of sperm or spermatozoa directly into a uterus.

The term “in vitro fertilization” or “IVF” refers to a process by which oocytes are fertilized by sperm outside of the body, in vitro. IVF is a major treatment in infertility when in vivo conception has failed.

The term “intracytoplasmic sperm injection” or “ICSI” refers to an in vitro fertilization procedure in which a single sperm is injected directly into the cytoplasm of an egg. This procedure is most commonly used to overcome male infertility factors, although it may also be used where oocytes cannot easily be penetrated by sperm, and occasionally as a method of in vitro fertilization.

As used herein, the term “enriched” refers to a composition or fraction or preparation wherein an object species has been partially purified such that the concentration of the object species is substantially higher than the naturally occurring level of the species in a finished product or preparation without enrichment.

The term “sperm capacitation” refers to the sperm having the ability to undergo acrosomal exocytosis and binding to and penetrating through the zona pellucida of an unfertilized egg. Completion of capacitation is manifested by the ability of sperm to bind to the zona pellucida and to undergo ligand-induced acrosomal reaction.

Some numerical values disclosed throughout are referred to as, for example, “X is at least or at least about 100; or 200 [or any numerical number].” This numerical value includes the number itself and all of the following:
i. Xis at least 100;
ii. X is at least 200;
iii. X is at least about 100; and
iv. X is at least about 200.

All these different combinations are contemplated by the numerical values disclosed throughout. All disclosed numerical values should be interpreted in this manner, whether it refers to an administration of a therapeutic agent or referring to days, months, years, weight, dosage amounts, etc., unless otherwise specifically indicated to the contrary.

The ranges disclosed throughout are sometimes referred to as, for example, “X is administered on or on about day 1 to 2; or 2 to 3 [or any numerical range].” This range includes the numbers themselves (e.g., the endpoints of the range) and all of the following:
i. X being administered on between day 1 and day 2;
ii. X being administered on between day 2 and day 3;
iii. X being administered on between about day 1 and day 2;
iv. X being administered on between about day 2 and day 3;
v. X being administered on between day 1 and about day 2;
vi. X being administered on between day 2 and about day 3;
vii. X being administered on between about day 1 and about day 2; and
viii. X being administered on between about day 2 and about day 3.

All these different combinations are contemplated by the ranges disclosed throughout. All disclosed ranges should be interpreted in this manner, whether it refers to an administration of a therapeutic agent or referring to days, months, years, weight, dosage amounts, etc., unless otherwise specifically indicated to the contrary.

It should be understood that for all numerical bounds describing some parameter in this application, such as “about,” “at least,” “less than,” and “more than,” the description also necessarily encompasses any range bounded by the recited values. Accordingly, for example, the description “at least 1, 2, 3, 4, or 5” also describes, inter alia, the ranges 1-2, 1-3, 1-4, 1-5, 2-3, 2-4, 2-5, 3-4, 3-5, and 4-5, et cetera.

Methods of Evaluating Sperm Quality

In one aspect a method for evaluating sperm quality of a sperm from a mammalian sperm donor is provided. The method comprises; incubating a sample of sperm obtained from said mammalian donor under energy depletion conditions for a time suitable to generate a potentiated sperm in the sample. The potentiated sperm is provided with an effective amount of a first energy source, and optionally an effective amount of a second energy source and a sperm function of the sperm in the sample is determined to evaluate sperm quality. In some embodiments, the sperm are identified to be a of a higher sperm quality. In some embodiments, higher quality sperm comprise an increase in sperm function. The increase in sperm function can be relative to a suitable control. As it relates to the present disclosure, the sperm identified to be of higher sperm quality is suitable, for example, for natural conception or a less invasive reproduction modality, such as artificial insemination (e.g., intracervical insemination or intrauterine insemination).

A sperm from a donor identified to be of higher sperm quality can result in successful fertilization of an egg leading to generation of an embryo that is able to give rise to pregnancy and thus suitable for a less invasive reproduction modality. Sperm quality can be determined by characteristics including but not limited to sperm viability, sperm count, sperm morphology and sperm function (e.g., motility, sperm capacitation). Sperm viability, sperm count, sperm morphology and sperm function (e.g., motility) are considered as markers of fertility and used to predict pregnancy success.

The methods disclosed herein are particularly suitable for determining whether a selected reproduction modality for the infertile donor or hypofertile donor or subfertile donor or sperm donor with difficulty to conceive for more than one year, shall be performed with a reasonable expectation of success. In case when the sperm sample from a donor is identified to comprise higher sperm quality, a less invasive reproduction modality comprising of intrauterine Insemination (IUI) or natural conception can be preferred. In case when the sperm sample from a donor is identified to comprise lower sperm quality, a more invasive reproduction modality of intracytoplasmic sperm injection (ICSI) or in vitro fertilization may be directly performed.

The methods disclosed herein can be useful for identifying a sperm sample comprising higher quality sperm from a plurality of sperm samples, prior to performing assisted reproduction methods or a particular reproduction modality. The methods therefore can be employed to select a superior sperm sample for use in assisted reproductive technologies, for example, from a sperm bank.

The methods disclosed herein can be applied for monitoring a treatment capable of increasing sperm function and thus improving sperm quality. Typically, said treatment may be a normozoospermia treatment. The “normozoospermia treatment” relate to any type of normozoospermia therapy undergone by the normozoospermic subjects previously to collecting the normozoospermic semen samples, including gonadotropin, Human Chorionic Gonadotropin (HCG), Human Menopausal Gonadotropin (HMG) and bromocryptine.

The method of the disclosure can be applied for monitoring a sterilization treatment (e.g., drug compounds) of a male subject. For example, the effectiveness of an agent to affect a sperm function and therefore sperm quality according to the disclosure can be monitored during treatments of subjects receiving sterilization treatments. The “sterilization treatment” relate to any type of sterilization therapy undergone by the male subjects, including pharmacological sterilization.

Sperm quality may be assessed by any method known in the art and methods described herein. Typically, microscopic assays are used to assess sperm concentration, motility and morphology. Sperm count can be estimated, for example, by kits that measure the amount of a sperm-associated protein. Sperm volume can be determined by measuring the weight of the sample. Sperm motility can be assessed, for example, by Computer Assisted Semen Analysis (CASA). Most CASA systems are based on image analysis, but alternative methods exist such as tracking cell movement on a digitizing tablet. CASA are most-often used for the assessment of sperm concentration and motility characteristics, such as linear velocity and curvilinear velocity. Further sperm function tests include, but are not limited to, a Hamster zona-free ovum test for determining ability of sperm to penetrate the oocyte, i.e., fertilization ability. Sperm Chromatin Structure Assay (SCSA) can be used for measuring DNA fragmentation or sperm-zona pellucida binding assay for determining increase in sperm capacitation.

Sperm Function

Provided herein are methods to evaluate sperm quality and methods to identify suitability of a sperm for a reproduction modality. The methods comprise incubating a sample of sperm obtained from the mammalian donor under energy depletion for a time suitable to potentiate the mammalian sperm, providing the potentiated mammalian sperm with an effective amount of a first energy source, such as (i) a glycolytic energy source or (ii) a gluconeogenesis substrate, and optionally providing the mammalian sperm from step (b) with an effective amount of a second energy source, selected from: (i) the glycolytic energy source or (ii) the gluconeogenesis substrate, wherein the energy source provided is not the one selected as first energy source, and determining a sperm function. In some embodiments, an increase in sperm function compared to a suitable control is indicative the sperm of the mammalian donor to be higher quality sperm. In some embodiments, an increase in sperm function relative to a suitable control is indicative of the sperm of the mammalian donor as suitable for a reproduction modality that is a less invasive reproduction modality. In some embodiments, a lack of increase in sperm function is indicative of the sperm to be suitable for a reproduction modality that is a more invasive reproduction modality. In some embodiments, the method is performed in vitro. Increased sperm function includes one or more of: increased motility such as the percentage of sperm in a population exhibiting hyperactivation and/or intermediate motility as assessed by CASAnova (see Goodson et al., 2017, Biol. Reprod. 97:698-708; doi:10.1093/biolre/iox120), increased autophagy, increased capacitation, and increased rates of fertilization, e.g., development to at least two cells, blastocyst development, or live birth. Accordingly, in some embodiments, sperm function can be sperm motility, curvilinear velocity, amplitude of lateral head displacement, autophagy, sperm capacitation, percentage of hyperactivated sperm, percentage of intermediate motility sperm and percentage of hyperactivated sperm and intermediate motility sperm, ability to fertilize an egg, generation of an embryo. In some embodiments, the embryo generated by the sperm with increased function comprises one or more characteristics selected from increased viability, increased implantation, increased ability to develop to a at least a 2-cell developmental stage, blastocyst developmental stage or an offspring, including an offspring with improved fitness, such as absence (or reduced incidence) of a condition such as obesity (or an obesity-associated disorder such as cancer, cardiovascular disease, infertility and the like.

In some embodiments, the first and second energy sources are provided in a serial manner (e.g., providing a first energy source and subsequently providing a second energy source). In some embodiments, the first and second energy sources are provided simultaneously. An increase in one or more sperm functions, as contemplated herein, constitutes an increase in the one or more sperm functions relative to a suitable control. In some embodiments, the one or more sperm functions can be increased by at least or at least about: 5%, 10%, 20%, 30%, 40%, 50%, 60%, 75%, 80%, 90%, or 100%, 200%, 300% or more. In some embodiments, the one or more sperm functions can be increased by from 10% to 200%, from 25% to 150%, from 50% to 100%, or from 70% to 90%.

In some embodiments, sperm activity and/or function encompass physiological processes such as, sperm motility, sperm tropism (namely, the tendency of sperm to move towards or away from certain stimuli), and ability to fertilize an egg. In some embodiments, activity and/or function can further include processes which occur prior to, during fertilization and/or interaction with the egg (or membranes/layers thereof)—such processes may include, for example sperm capacitation and acrosomal activity, and/or processes after fertilization of egg, for example, formation of an embryo. In some embodiments, the embryo exhibits increased (longer) viability, improved implantation, and/or ability to develop to a 2-cell stage, a blastocyst, or to an offspring resulting in live birth.

Exemplary methods to determine an increase in sperm function can be by motility, mucus penetration, oocyte fertilization or subsequent embryonic development and the like. Methods to determine sperm function are well known in the art, see for example, SS. Vasan Indian J Urol. 2011 January-March; 27(1): 41-48. Methods for determining an increase in sperm function are known to one of skill in the art. See, for example, PCT/US2019/063687, the contents of which are incorporated herein by reference, in its entirety.

Sperm Motility

In some embodiments, sperm function comprises sperm motility. In some embodiments, determining sperm function comprises determining sperm motility. With regard to sperm motility, one of skill will appreciate that the term “motility” not only relates to general movement, but may be applied to other aspects of motility such as, for example, the speed of movement of a sperm cell and/or any increase or decrease in the proportion of moving sperm cells in any given population. As such, the determining sperm function is not limited to determining sperm motility, but can include determining the speed of movement of a sperm cell and/or the proportion (percentage) of moving cells in any given population of sperm. As such, an increase in sperm function can comprise an increase in sperm motility, increase in speed of movement of a sperm cell, increase in proportion of moving cells in any given population, or a combination thereof.

Motility of sperm can be expressed as the total percent of motile sperm, or the velocity of sperm that are motile. These measurements may be made by a variety of assays, but are conveniently assayed in one of two ways. Either a subjective visual determination is made using a phase contrast microscope when the sperm are placed in a hemocytometer or on a microscope slide, or a computer assisted semen analyzer is used. Under phase contrast microscopy, motile and total sperm counts are made and speed is assessed as fast, medium or slow. A second method of assessing sperm motility is by using a computer assisted semen analyzer (Hamilton Thorn, Beverly, Mass.), the motility characteristics of individual sperm cells in a sample are objectively determined. Briefly, a sperm sample is placed onto a slide or chamber designed for the analyzer. The analyzer tracks individual sperm cells and determines motility and velocity of the sperm. Data is expressed as percent motile, and measurements are obtained for path velocity and track speed as well.

Accordingly, the term “motility” encompasses percentage of motile sperm which can be the percentage of the total number of sperm assessed that fall within all World Health Organization (WHO) categories of motility except the category designated “no motility” regardless of velocity or directionality as discussed in Cooper et al. Human Reproduction update, Vol 16, No 3 pp 231-245, 2010. Manual counting classifies sperm cells into 4 categories (immotile, locally motile, nonlinear and linear motile) using qualitative subjective criteria of selection.

The term “motility” encompasses percentage of motile sperm, i.e., the percentage of total number of sperm assessed in a population exhibiting progressive motility, hyperactivated motility and/or intermediate motility based on Computer assisted sperm analysis (For example, as assessed by CASAnova; see Goodson et al, 2017, Biol. Reprod. 97:698-708).

In some embodiments, an increase in sperm function comprises an increase in percentage of progressive motility sperm, i.e., percentage of sperm exhibiting linear movement from one point to another, with turns of the head of less than 90 degrees from sperm that are otherwise non-progressive, i.e., sperm that move but do not make forward progression. In some embodiments, an increase in sperm function comprises an increase in percentage of intermediate motility sperm. Intermediate motility sperm is characterized by movement that is similar to progressive vigorous motility, but has a larger variance from the path and turns of the sperm head of approximately 90 degrees, such as an oscillating movement. In some embodiments, the increase in sperm function comprises an increase in percentage of activated hyperactive sperm, also known as hyperactivated sperm. Hyperactivated sperm motility is characterized by sperm that have a high amplitude, asymmetrical beating pattern of the flagellum. Hyperactivated motility is characterized by vigorous movement with many seemingly random variations without a well-defined progressive path and turns of the sperm head of greater than 90 degrees. Hyperactivated sperm motility is more vigorous and short term than progressive motility. Biologically, hyperactivated sperm motility is important to enable sperm to traverse the egg outer investments prior to fertilizing the mature egg. In some embodiments, the increase in sperm function comprises an increase in percentage of hyperactivated sperm and intermediate motility sperm in a given population of sperm e.g., in a given sample of sperm. In related embodiments, the step of determining sperm function comprises calculating a ratio of percentage of hyperactivated sperm and intermediate motility sperm in a sample of sperm to percentage of hyperactivated sperm and intermediate motility sperm in a suitable control. In some embodiments, the increase in sperm function is indicated by the ratio of percentage of hyperactivated sperm and intermediate motility sperm in a sample of sperm to percentage of hyperactivated sperm and intermediate motility sperm in a suitable control. In some embodiments, the ratio is greater than 1, e.g., greater than about: 1.05, 1.1, 1.2, 1.25, 1.3, 1.35, 1.4, 1.45, 1.5, 1.55, 1.6, 1.65, 1.7, 1.75, 1.8, 1.85, 1.9, 1.95, or more, e.g., greater than about: 2, 3, 4, 5, 6, 7, 8, 9, or 10. In some embodiments, the lack of increase in the sperm function is indicated by the ratio of percent of hyperactivated sperm and intermediate motility sperm in the sperm sample to percentage of hyperactivated sperm and intermediate motility sperm in a suitable control, optionally wherein the ratio is 1 or lesser, e.g., less than about: 0.99, 0.95, 0.9, 0.85, 0.8, 0.75, 0.7 or less.

It should be understood that other standardized measures of sperm motility parameters can also be used. Other measures of sperm motility include “velocity” and “linearity” which can be assessed using automatic semen analyzers. In some embodiments, determining a sperm function comprises determining sperm motility by average path velocity (VAP), straight-line velocity (VSL), curvilinear velocity (VCL), amplitude of lateral head displacement (ALH) and beat cross frequency (BCF) or other movement parameters of the sperm including parameters known to those of skill in the art. Accordingly, in some embodiments, the increase in sperm function can comprise increase in average path velocity (VAP), straight-line velocity (VSL), curvilinear velocity (VCL), amplitude of lateral head displacement (ALH) and beat cross frequency (BCF) or other movement parameters of the sperm including parameters known to those of skill in the art. Curvilinear velocity (VCL) is the measure of the rate of travel of the centroid of the sperm head over a given time period. Average path velocity (VAP) is the velocity along the average path of the spermatozoon. Straight-line velocity (VSL) is the linear or progressive velocity of the cell. Linearity of forward progression (LIN) is the ratio of VSL to VCL and is expressed as percentage. Amplitude of lateral head displacement (ALH) of the sperm head is calculated from the amplitude of its lateral deviation about the cell's axis of progression or average path. Methods of measuring sperm motility by CASA are well known in the art, see for example, WO2012061578A2. An increase in sperm motility, as contemplated herein, constitutes an increase in the motility of sperm relative to a suitable control.

In some embodiments, sperm motility is measured by the CASA system. In some embodiments, the motility of the sperm is classified by the CASA system as weak, slow, progressive, intermediate, or hyperactivated. In some embodiments, the hyperactivated sperm is classified by the CASA system as sperm with a VCL ≥150 μm/s, a LIN ≤50%, and an ALH >7 μm when analyzed at 60 Hz. In some embodiments, sperm motility is classified according to the criteria in Goodson et al. (2017), Biol. Reprod. 97:698-708.

In some embodiments, the increase in sperm motility can be more than about: 10%, 20%, 30%, 40%, 50%, 60%, 70%, 80%, 90%, or 99% relative to a suitable control. In some embodiments, the increase in sperm motility can be at least 5%, at least 10%, at least 20%, at least 30%, at least 40%, at least 50%, at least 60%, at least 70%, at least 80%, at least 90%, or at least 95%. In some embodiments, the increase in sperm motility can be by a factor of at least 10, at least 100, at least 1,000, at least 10,000. In some embodiments, the sperm motility can be increased by from 10% to 200%, from 25% to 150%, from 50% to 100%, or from 70% to 90%. In some embodiments, an increase in sperm function or an increase in sperm motility can be an increase in percentage of hyperactivated sperm. In some embodiments, an increase in sperm function or an increase in sperm motility can be an increase in percentage of intermediate motility sperm. In some embodiments, an increase in sperm function or an increase sperm motility can be an increase in percentage of progressive motility sperm. In some embodiments, an increase in sperm function or an increase in sperm motility can be an increase in percentage of the hyperactivated sperm and intermediate motility sperm. In some embodiments, the level of hyperactivated sperm, progressive motility sperm, intermediate motility sperm or a combination thereof is increased so that hyperactivated sperm, progressive motility sperm, intermediate motility sperm or a combination thereof comprise at least about: 5%, 5.5%, 6.0%, 6.5%, 7.0%, 7.5%, 8.0%, 8.5%, 9.0%, 9.5%, 10.0%, 10.5%, 11.0%, 11.5%, 12.0%, 12.5%, 13.0%, 13.5%, 14.0%, 14.5%, 15.0%, 15.5%, 16.0%, 16.5%, 17.0%, 17.5%, 18.0%, 18.5%, 19.0%, 19.5%, 20.0%, 20%, 25%, 30%, 35%, 40%, 50% or more of the total sperm in a sample of sperm. An increase in sperm motility is indicative of increased sperm function. Accordingly, an increase in sperm motility is indicative of higher sperm quality. In some embodiments, an increase in sperm motility in a sample of sperm obtained from a mammalian donor, identifies the donor's sperm to be suitable for a less invasive reproduction modality. In some embodiments, the lack of increase in sperm motility in a sample from a mammalian donor, identifies donor's sperm as suitable for a more invasive reproduction modality.

Sperm Capacitation

In some embodiments, a sperm function comprises sperm capacitation. In some embodiments, determining a sperm function comprises determining sperm capacitation. In some embodiments, an increase in sperm function comprises an increase in sperm capacitation. Completion of capacitation is manifested by the ability of sperm to bind to the zona pellucida and to undergo ligand-induced acrosomal reaction. Methods to determine sperm capacitation are known in the art, for example, the most common sperm-zona pellucida binding tests currently utilized are the hemizona assay (or HZA) and a competitive intact-zona binding assay. A hemizona assay measures the ability of sperm to undergo capacitation and bind to an oocyte. Sperm is incubated with dead oocytes which are surrounded by the zona pellucida, an acellular coating of oocytes. Capacitated sperm bind to the zona and the number of sperm binding is counted microscopically. This number correlates with the number of normal capacitated sperm in a sample and with fertility of a sperm sample. For example, see Cross N L et al. Gamete Res. 1986; 15:213-26.

In some embodiments, an increase in sperm capacitation can be more than about: 10%, 20%, 30%, 40%, 50%, 60%, 70%, 80%, 90%, or 99% relative to a suitable control. In some embodiments, an increase in sperm capacitation can be at least 5%, at least 10%, at least 20%, at least 30%, at least 40%, at least 50%, at least 60%, at least 70%, at least 80%, at least 90%, or at least 95%. In some embodiments, an increase in sperm capacitation can be by a factor of at least 10, at least 100, at least 1,000, at least 10,000. In some embodiments, the sperm capacitation can be increased by from 10% to 200%, from 25% to 150%, from 50% to 100%, or from 70% to 90%. In some embodiments, the level of sperm capacitation is increased so that capacitated sperm can comprise at least about: 5%, 5.5%, 6.0%, 6.5%, 7.0%, 7.5%, 8.0%, 8.5%, 9.0%, 9.5%, 10.0%, 10.5%, 11.0%, 11.5%, 12.0%, 12.5%, 13.0%, 13.5%, 14.0%, 14.5%, 15.0%, 15.5%, 16.0%, 16.5%, 17.0%, 17.5%, 18.0%, 18.5%, 19.0%, 19.5%, 20.0%, 20%, 25%, 30%, 35%, 40%, 50%, or more, of the total sperm in a sample of sperm. An increase in sperm capacitation is indicative of increase in sperm function. Accordingly, an increase in sperm capacitation is indicative of higher sperm quality. In some embodiments, an increase in sperm capacitation in a sample of sperm obtained from a mammalian donor, identifies the donor's sperm to be suitable for a less invasive reproduction modality. In some embodiments, the lack of increase in sperm capacitation in a sample obtained from a mammalian donor, identifies the donor's sperm as suitable for a more invasive reproduction modality.

Fertilizing Ability

In some embodiments, the sperm identified to be of higher sperm quality comprises increased ability of fertilizing an egg. In some embodiments, the sperm function comprises ability of the sperm to fertilize an egg. In some embodiments, determining a sperm function comprises determining the ability of sperm to fertilize an egg. In some embodiments, an increase in sperm function comprises an increase in the ability of the sperm to fertilize an egg. The fertilizing ability of a sperm can be determined, for example, by a sperm penetration assay. The spermatozoa penetration assay (SPA) utilizes the golden hamster egg, which is unusual in that removal of its zona pellucida results in loss of all species specificity to egg penetration. This test is conducted to determine the ability of sperm to penetrate into the oocyte (Rogers et al., Fert. Ster. 32:664, 1979). Briefly, commercially available zona free hamster oocytes can be used (Fertility Technologies, Natick, Mass.). Hamster oocytes are suitable in this assay for sperm of any species. Sperm are incubated for 3 hours with the hamster oocytes. Following incubation, oocytes are stained with acetolacmoid or equivalent stain and the number of sperm penetrating each oocyte is counted microscopically. Another parameter of sperm fertilizing ability is the ability to penetrate cervical mucus. This penetration test can be done either in vitro or in vivo. Briefly, in vitro, a commercial kit containing cervical mucus (Tru-Trax, Fertility Technologies, Natick, Mass.), typically bovine cervical mucus, is prepared. Sperm are placed at one end of the track and the distance that sperm have penetrated into the mucus after a given time period is determined. Alternatively, sperm penetration of mucus may be measured in vivo in women. In some embodiments, the increase in fertilizing ability can be more than about: 10%, 20%, 30%, 40%, 50%, 60%, 70%, 80%, 90%, or 99% relative to a suitable control. In some embodiments, the increase in fertilizing ability can be at least 5%, at least 10%, at least 20%, at least 30%, at least 40%, at least 50%, at least 60%, at least 70%, at least 80%, at least 90%, or at least 95%. In some embodiments, the increase in fertilizing ability can be by a factor of at least 10, at least 100, at least 1,000, at least 10,000. In some embodiments, the fertilizing ability can be increased by from 10% to 200%, from 25% to 150%, from 50% to 100%, or from 70% to 90%. In some embodiments, the level of fertilizing ability is increased so that the number of sperm able to fertilize an egg is at least about: 5%, 5.5%, 6.0%, 6.5%, 7.0%, 7.5%, 8.0%, 8.5%, 9.0%, 9.5%, 10.0%, 10.5%, 11.0%, 11.5%, 12.0%, 12.5%, 13.0%, 13.5%, 14.0%, 14.5%, 15.0%, 15.5%, 16.0%, 16.5%, 17.0%, 17.5%, 18.0%, 18.5%, 19.0%, 19.5%, 20.0%, 20%, 25%, 30%, 35%, 40%, 50% or more of the total sperm in a sperm sample.

An increase in fertilizing ability is indicative of increase in sperm function. Accordingly, an increase in fertilizing ability is indicative of higher sperm quality. In some embodiments, an increase in fertilizing ability in a sample of sperm obtained from a mammalian donor, identifies the donor's sperm to be suitable for a less invasive reproduction modality. In some embodiments, the lack of increase in fertilizing ability in a sample obtained from a mammalian donor, identifies the donor's sperm as suitable for a more invasive reproduction modality.

Autophagy

In some embodiments, a sperm function comprises autophagy. In some embodiments, determining a sperm function comprises determining autophagy. In some embodiments, the increase in sperm function comprises an increase in autophagy. Methods to determine an increase in autophagy are known in the art. For example, an increase in autophagy can be determined by increase in one or more of autophagy marker proteins. The detection of increase in marker protein can be done by conventional methods such as immunoblotting. Non-limiting examples of autophagy marker proteins include, Atg 5, Atg 16, p62, LC3-II, AMPK, m-TOR and Beclin 1. LC3-II has been widely used to study autophagy and it has been considered as an autophagosomal marker in mammals. A ratio of LC3-II/LC3-I can be used as a determinant of increase in autophagy. An increase in levels of one or more autophagy marker proteins (e.g., Atg 5, Atg 16, p62 and LC3-II, AMPK, m-TOR and Beclin 1), and/or an increase in ratio of LC3-II/LC3-I can be indicative of increase in sperm function.

In some embodiments, the increase in autophagy can be more than about: 10%, 20%, 30%, 40%, 50%, 60%, 70%, 80%, 90%, or 99% relative to a suitable control. In some embodiments, the increase in sperm autophagy can be at least 5%, at least 10%, at least 20%, at least 30%, at least 40%, at least 50%, at least 60%, at least 70%, at least 80%, at least 90%, or at least 95%. In some embodiments, the increase in sperm autophagy can be by a factor of at least 10, at least 100, at least 1,000, at least 10,000. In some embodiments, the sperm autophagy can be increased by from 10% to 200%, from 25% to 150%, from 50% to 100%, or from 70% to 90%. In some embodiments, the level of sperm autophagy is increased so that sperm with increased autophagy can comprise at least about: 5%, 5.5%, 6.0%, 6.5%, 7.0%, 7.5%, 8.0%, 8.5%, 9.0%, 9.5%, 10.0%, 10.5%, 11.0%, 11.5%, 12.0%, 12.5%, 13.0%, 13.5%, 14.0%, 14.5%, 15.0%, 15.5%, 16.0%, 16.5%, 17.0%, 17.5%, 18.0%, 18.5%, 19.0%, 19.5%, 20.0%, 20%, 25%, 30%, 35%, 40%, 50%, or more, of the total sperm in a given sperm sample. An increase in sperm autophagy is indicative of increase in sperm function. In some embodiments, an increase in autophagy can be indicated by a reduction in cellular RNA levels (such as small non-coding RNAs, including microRNA). In some embodiments, the hyperactivated (or intermediate motility, or hyperactivated and intermediate motility) sperm in a preparation undergoing autophagy are classified by at least about: 10, 15, 20, 25, 30, 35, 40, 45, 50%, or more (e.g., 2, 3, 4, 5, 6, 7, 8, 9, 10-fold, or more) reduction in intracellular RNA concentration (such as small non-coding RNAs, including microRNA), relative to a suitable control. An increase in autophagy is indicative of increase in sperm function. Accordingly, an increase in autophagy is indicative of higher sperm quality. In some embodiments, an increase in autophagy in a sample of sperm obtained from a mammalian donor, identifies the donor's sperm to be suitable for a less invasive reproduction modality. In some embodiments, the lack of increase in autophagy in a sample obtained from a mammalian donor, identifies the donor's sperm as suitable for a more invasive reproduction modality.

Generating Embryos

In some embodiments, a sperm function comprises generating an embryo, wherein the embryo exhibits longer viability improved implantation, and/or ability to develop to at least a 2 cell developmental stage, or blastocyst developmental stage relative to an embryo generated by a suitable control. In some embodiments, determining a sperm function comprises determining generation of an embryo, wherein the embryo exhibits longer viability improved implantation, and/or ability to develop to at least a 2 cell developmental stage, or blastocyst developmental stage relative to an embryo generated by a suitable control. In some embodiments, an increase in sperm function comprises a generation of an embryo, wherein the embryo exhibits longer viability improved implantation, and/or ability to develop to at least a 2 cell developmental stage, or blastocyst developmental stage relative to an embryo generated by a suitable control.

In the context of this specification, the terms “embryo with increased viability” and “embryo with longer viability” mean an increase or enhancement in the likelihood of survival of an embryo(s) which has been generated by the sperm sample of the methods or a sperm evaluated by the methods disclosed herein prior to providing access to an egg, compared to the likelihood of survival of an embryo(s) which has been generated by a suitable control. In some embodiments, the embryo is generated by an assisted reproductive technology e.g., IVF or ICSI. In some embodiments, the embryo is generated in vivo in the reproductive tract of a female mammalian subject by artificial insemination, e.g., intrauterine insemination.

For the purposes of the present disclosure, an embryo viability may be reflected in a number of indicators. For example, increased embryo viability may result in increased embryo implantation rates following fertilization, decreased pre- and post-implantation embryo lethality, increased clinical pregnancy rates or increased birth rates. The embryo viability can refer to viability of an embryo in vitro or in vivo.

In some embodiments, the increase in viability of embryo generated by sample of sperm can be more than about: 10%, 20%, 30%, 40%, 50%, 60%, 70%, 80%, 90%, or 99% relative to an embryo generated by a suitable control. In some embodiments, the increase in embryo viability can be at least 5%, at least 10%, at least 20%, at least 30%, at least 40%, at least 50%, at least 60%, at least 70%, at least 80%, at least 90%, or at least 95%. In some embodiments, the increase in embryo viability can be by a factor of at least 10, at least 100, at least 1,000, at least 10,000. In some embodiments, the embryo viability can be increased by from 10% to 200%, from 25% to 150%, from 50% to 100%, or from 70% to 90%. In some embodiments, the level of sperms that can generate an embryo with increased viability is at least about: 5%, 5.5%, 6.0%, 6.5%, 7.0%, 7.5%, 8.0%, 8.5%, 9.0%, 9.5%, 10.0%, 10.5%, 11.0%, 11.5%, 12.0%, 12.5%, 13.0%, 13.5%, 14.0%, 14.5%, 15.0%, 15.5%, 16.0%, 16.5%, 17.0%, 17.5%, 18.0%, 18.5%, 19.0%, 19.5%, 20.0%, 20%, 25%, 30%, 35%, 40%, 50% or more of the total sperm sample. In some embodiments, generation of an embryo with increased viability is indicative of higher sperm quality. In some embodiments, generation of an embryo with increased viability, identifies the donor's sperm to be suitable for a less invasive reproduction modality. In some embodiments, the lack of generation of an embryo with increased viability, identifies the donor's sperm as suitable for a more invasive reproduction modality.

In some embodiments, a sperm function comprises generation of an embryo by the sample of sperm with increased ability to develop through normal developmental stages (e.g., 2 cell stage, blastocyst stage, development into an offspring and live birth) relative to an embryo generated by a suitable control. In some embodiments, increase in rate of an embryo progressing through normal developmental stages, generated by the sample of sperm can be more than about: 10%, 20%, 30%, 40%, 50%, 60%, 70%, 80%, 90%, or 99% relative to an embryo generated by suitable control. In some embodiments, the increase in rate of an embryo progressing through normal developmental stages can be at least 5%, at least 10%, at least 20%, at least 30%, at least 40%, at least 50%, at least 60%, at least 70%, at least 80%, at least 90%, or at least 95%. In some embodiments, the increase in rate of an embryo progressing through normal developmental stages can be by a factor of at least 10, at least 100, at least 1,000, at least 10,000. In some embodiments, the rate of embryo progressing through normal developmental stages can be increased by from 10% to 200%, from 25% to 150%, from 50% to 100%, or from 70% to 90%. In some embodiments, the level of sperms that can generate an embryo with ability to progress through normal developmental stages is at least about: 5%, 5.5%, 6.0%, 6.5%, 7.0%, 7.5%, 8.0%, 8.5%, 9.0%, 9.5%, 10.0%, 10.5%, 11.0%, 11.5%, 12.0%, 12.5%, 13.0%, 13.5%, 14.0%, 14.5%, 15.0%, 15.5%, 16.0%, 16.5%, 17.0%, 17.5%, 18.0%, 18.5%, 19.0%, 19.5%, 20.0%, 20%, 25%, 30%, 35%, 40%, 50% or more of the total sperm in a sample. Generation of an embryo with ability to progress through one or more normal developmental stages is indicative of increase in sperm function or of higher sperm quality.

In some embodiments, increase in sperm function comprises generation of an embryo with improved implantation rate or improved rate of pregnancy by the sample of sperm relative to embryo generated by suitable control. In some embodiments, the increase in implantation rate of an embryo generated by the sperm sample or pregnancy rate upon implantation of an embryo can be more than about: 10%, 20%, 30%, 40%, 50%, 60%, 70%, 80%, 90%, or 99% relative to an embryo generated by a suitable control. In some embodiments, the increase in an embryo implantation rate or pregnancy rate can be at least 5%, at least 10%, at least 20%, at least 30%, at least 40%, at least 50%, at least 60%, at least 70%, at least 80%, at least 90%, or at least 95%. In some embodiments, the increase in rate of embryo implantation or rate of pregnancy can be by a factor of at least 10, at least 100, at least 1,000, at least 10,000. In some embodiments, the embryo implantation or pregnancy rate can be increased by from 10% to 200%, from 25% to 150%, from 50% to 100%, or from 70% to 90%. In some embodiments, the level of sperms that can generate an embryo with increased implantation rate or improved pregnancy rate is at least about: 5%, 5.5%, 6.0%, 6.5%, 7.0%, 7.5%, 8.0%, 8.5%, 9.0%, 9.5%, 10.0%, 10.5%, 11.0%, 11.5%, 12.0%, 12.5%, 13.0%, 13.5%, 14.0%, 14.5%, 15.0%, 15.5%, 16.0%, 16.5%, 17.0%, 17.5%, 18.0%, 18.5%, 19.0%, 19.5%, 20.0%, 20%, 25%, 30%, 35%, 40%, 50% more of the total sperm in a sample. Generation of embryos with improved implantation (i.e., increased rate of implantation) or increased pregnancy rate upon implantation is indicative of increase in sperm function or of higher sperm quality.

Methods of Identifying Reproduction Modality

In one aspect, provided herein is a method for identifying whether a sperm from a prospective sperm donor is suitable for a reproduction modality. In some embodiments, the method is useful as a screening method for identifying mammalian sperm donors who will have a high probability of successful fertilization resulting in embryo generation and pregnancy (in some embodiments, success may be defined as live birth), such as when using a particular reproduction modality. In another aspect, a method for identifying a suitable reproduction modality for a sperm is provided. In some embodiments, the method is useful to inform selection of a reproduction modality for a given sperm sample or a given donor's sperm.

The methods comprise: incubating a sample of sperm obtained from a mammalian donor under energy depletion conditions for a time suitable to potentiate the sperm in the sample, providing the potentiated sperm with an effective amount of a first energy source and optionally providing an effective amount of a second energy source, and determining a sperm function in the sample. In case, when the sperm function is determined to be increased relative to a suitable control, the donor's sperm is identified as suitable for a less invasive reproduction modality. In some embodiments, where a lack of increase in a sperm function is determined relative to a suitable control, the sperm of the donor is identified as suitable for a more invasive reproduction modality.

In one aspect, the method disclosed herein is useful for identifying a reproduction modality suitable for a given donor's sperm or for a given sperm sample. For instance, in some embodiments, an increase in a sperm function in a sample obtained from a donor identifies a less invasive reproduction modality as suitable for the given sample and for the donor's sperm. In some embodiments, where a lack of increase in a sperm function is determined in a sample obtained from a donor, a more invasive reproduction modality is identified as suitable for the given sample and for the donor's sperm.

In another aspect, a method is provided that is more highly predictive of successful fertilization resulting in embryo generation and pregnancy when using an identified reproduction modality. Accordingly, the methods disclosed herein can be prognostic to identify likelihood of success of a suitable reproduction modality when using a sperm sample from a donor. Thus, in some aspects, the present disclosure describes a screening method that can predict outcome of a reproduction modality when using a sperm from a potential mammalian donor. By way of example, the choices between reproduction modality of natural conception, ICSI, IUI, or IVF can be made based on the evaluation of a sperm function in donor's sample in the described methods.

In some embodiments, the method further comprises, responsive to a determination of lower sperm quality, contacting sperm from the donor (e.g., sperm from the same ejaculate as the evaluated sperm, or sperm from a different ejaculate) with media suitable for a more-invasive reproductive modality and/or providing the sperm from the donor with access to an egg by a more-invasive reproductive modality. In some embodiments, the determination of lower sperm quality comprises determining the % HI that is less than or equal to 75% of the % HI of the suitable control.

In some embodiments, the method further comprises, responsive to a determination of higher sperm quality, contacting sperm from the donor (e.g., sperm from the same ejaculate as the evaluated sperm, or sperm from a different ejaculate) with media suitable for a less-invasive reproductive modality, and/or providing the sperm from the donor with access to an egg by a less-invasive reproductive modality. In some embodiments, the determination of higher sperm quality comprises determining the % HI that is greater than or equal to 75% of the % HI of the suitable control. In some embodiments, the determination of higher sperm quality comprises determining the % HI that is greater than or equal to 125% of the % HI of the suitable control.

Methods of Reproduction

In one aspect, provided herein is a method of reproduction or a reproduction modality wherein a mammalian sperm is provided access to an egg, wherein prior to providing access, a sample of the mammalian sperm is evaluated to be of higher sperm quality or a suitable reproduction modality is identified for the sperm using the methods disclosed herein. The sperm evaluated to be of higher sperm quality or suitable for a selected reproduction modality will result in successful generation of an embryo. In some embodiments, the sperm of a mammalian donor or a sperm of a given sample is identified to be suitable for a less invasive reproduction modality. In some embodiments, the sperm of a mammalian donor or a sperm of a given sample is identified to be suitable for a more invasive reproduction modality.

In some embodiments, the reproduction method comprises: providing a sperm from a mammalian donor or a given sperm sample, that has been evaluated using methods disclosed herein to be of higher quality or suitable for a select reproduction modality, access to an egg. In some embodiments, the access to an egg is provided in vitro, thereby generating an embryo in vitro (e.g., more invasive reproduction modality). In some embodiments, the access to an egg is provided in vivo (e.g., less invasive reproduction modality), thereby generating an embryo in vivo. In some embodiments, the reproduction method comprises prior to providing access to an egg; incubating a sperm from a mammalian donor or a given sperm sample (e.g., a mammalian donor or a given sperm sample whose sperm has been evaluated to be of higher quality or suitable for a select reproduction modality) under energy depletion condition to potentiate the sperm. In some embodiments, the potentiated sperm is further provided an effective amount of a first energy source and optionally a second energy source prior to providing access to an egg. In some embodiments, the sperm which has been incubated under energy depletion conditions and provided with first energy source is inseminated in the reproductive tract of a female subject such that providing the second energy source and providing access to an egg to generate an embryo occurs in vivo.

In some embodiments, where the embryo is generated in vitro, the embryo can be cryopreserved for later use or can be further cultured in vitro to enable embryonic development. In some embodiments, the embryo is developed to at least a two-cell stage prior to cryopreserving and/or implantation into a female subject. In some embodiments, the embryo is developed to a developmental stage greater than the two-cell stage in vitro prior to further processing. In some embodiments, the embryo is developed to a blastocyst stage in vitro prior to further processing (e.g., cryopreservation or implantation into a female subject to develop into a full offspring). For in vitro incubation and culture of embryos during via assisted reproductive technologies (ART) procedures, a range of suitable media are available, the types and compositions of which are well known to those of skill in the art. Preferably the culture medium contains at least water, salts, nutrients, essential amino acids, vitamins and hormones, and may also include one or more growth factors. A variety of suitable culture media is commercially available, for example Earle's media, Ham's F10 media and human tubal fluid (HTF) media. The present disclosure also contemplates the co-culture in vitro of embryos on a layer of ‘feeder cells’ by methods known in the art. Appropriate ‘feeder cells’ for co-culture may include, for example, bovine oviductal cells or human tubal epithelial cells.

Those of skill in the art will appreciate that the advantages offered by the sperm evaluated by the methods disclosed herein are not limited to increasing probability of successful fertilization. Rather the sperm evaluated by methods herein can be useful to promote fertilization, whether the embryos are produced in vitro via assisted reproductive technologies (ART) or in the reproductive tract of the animal. The sperm evaluated by methods of the present disclosure provides improved fertilization, embryo viability, embryo implantation and pregnancy rates in assisted or otherwise unassisted pregnancies. Reproduction methods of present disclosure using sperm evaluated by methods herein are also useful for improved fertilization outcomes of sperm in male animals.

The embryo generated using a sperm evaluated to be of higher sperm quality or using a suitable reproduction modality identified by methods herein can exhibit longer viability, improved implantation, and/or ability to develop to at least a 2 cell developmental stage, or blastocyst development, or an offspring relative to an embryo generated by a sperm evaluated to be of lower sperm quality or unsuitable for a selected reproduction modality by methods disclosed herein. The methods herein are useful for preventing apoptosis or retarded development in embryos and for increasing pregnancy rates in animals.

In some embodiments, the increase in viability of embryo can be more than about: 10%, 20%, 30%, 40%, 50%, 60%, 70%, 80%, 90%, or 99%. In some embodiments, the increase in embryo viability can be at least 5%, at least 10%, at least 20%, at least 30%, at least 40%, at least 50%, at least 60%, at least 70%, at least 80%, at least 90%, or at least 95%. In some embodiments, the increase in embryo viability can be by a factor of at least 10, at least 100, at least 1,000, at least 10,000. In some embodiments, the embryo viability can be increased by from 10% to 200%, from 25% to 150%, from 50% to 100%, or from 70% to 90%.

Typically, the cleavage stage of embryo occurs during the first three days of culture. The in vitro generated embryo is transferred to a female subject by embryo transfer. “Embryo transfer” is the procedure in which one or more embryos and/or blastocysts are placed into the uterus or fallopian tubes. In the traditional IVF process, embryos are transferred to the uterine cavity two days after fertilization when each embryo is at the four (4) cell stage or three days after fertilization when the embryo is at the eight (8) cell stage. It has been recognized that it may be desirable to use embryos at the blastocyst stage when reached at day five to seven of culture. The present disclosure allows for embryo transfer at any time along the spectrum of embryo/blastocyst development. Through visual observation, such as by with the use of microscopy, blastocysts or embryos are considered ready to be transferred to the uterus when the blastocoel cavity is clearly evident and comprises greater than 50% of the volume of the embryo. In an in vivo environment, this stage would normally be achieved four to five days after fertilization, soon after the embryo has traversed the fallopian tube and arrives in the uterus. Embryonic developmental stage can be determined by visual observation of the embryo using microscopy (for example, Nikon Eclipse TE 2000-S microscope), the embryo will display certain determined physical or morphological features simultaneously before it is implanted into the uterus. The state of blastocyst maturity will be determined to be the range II AB-VI AA according to classification of Gardner et al, 1998.

The reproduction methods disclosed herein result in generation of embryos by the sperm evaluated by methods herein, with increased rate of progressing to 2-cell developmental stage, blastocyst developmental stage, or development to an offspring and live birth. In some embodiments, increase in rate of an embryo progressing through normal developmental stages, can be more than about: 10%, 20%, 30%, 40%, 50%, 60%, 70%, 80%, 90%, or 99%. In some embodiments, the increase in rate of an embryo progressing through normal developmental stages can be at least 5%, at least 10%, at least 20%, at least 30%, at least 40%, at least 50%, at least 60%, at least 70%, at least 80%, at least 90%, or at least 95%. In some embodiments, the increase in rate of an embryo progressing through normal developmental stages can be by a factor of at least 10, at least 100, at least 1,000, at least 10,000. In some embodiments, the rate of embryo progressing through normal developmental stages can be increased by from 10% to 200%, from 25% to 150%, from 50% to 100%, or from 70% to 90%. Generation of an embryo with ability to progress through one or more normal developmental stages is indicative of increased fertilization rate.

In vivo, an embryo attaches or implants to a wall of the uterus, creates a placenta, and develops into a fetal offspring during gestation until childbirth. Testing to determine whether one or more embryos have implanted into the endometrium, i.e., whether the procedure has resulted in successful pregnancy inception, is performed two weeks after transfer using blood tests on b-hCG (human chorionic gonadotropin), for example, and other techniques commonly known in the art. U.S. Pat. No. 4,315,908 to Zer et al. sets forth a method for detecting hCG in the urine by radioimmunoassay. U.S. Pat. No. 8,163,508 to O'Connor et al. provides a method and a kit for predicting pregnancy in a subject by hCG method by determining the amount of an early pregnancy associated isoform of hCG in a sample. Such methods of diagnosis and others are useful within the scope of the disclosure.

In some embodiments, the generated embryo using the reproduction methods disclosed herein exhibit improved implantation rate or improved rate of pregnancy. In some embodiments, the increase in implantation rate can be more than about: 10%, 20%, 30%, 40%, 50%, 60%, 70%, 80%, 90%, or 99% relative to an embryo generated by a suitable control. In some embodiments, the increase in an embryo implantation rate or pregnancy rate can be at least 5%, at least 10%, at least 20%, at least 30%, at least 40%, at least 50%, at least 60%, at least 70%, at least 80%, at least 90%, or at least 95%. In some embodiments, the increase in rate of embryo implantation or rate of pregnancy can be by a factor of at least 10, at least 100, at least 1,000, at least 10,000. In some embodiments, the embryo implantation or pregnancy rate can be increased by from 10% to 200%, from 25% to 150%, from 50% to 100%, or from 70% to 90%.

The sperm evaluated by methods disclosed herein can be applied in IVF, ICSI, artificial insemination (e.g., intra-uterine insemination) in human as well as in the biomedical research industry of animal models for human diseases (infertility, sperm dysfunction), and in the breeding and agricultural industries. The sperm can be provided access to an unfertilized egg of the same species as the sperm to promote in vitro fertilization, ICSI, or can be used for artificial insemination, including for example, intrauterine insemination of female subjects of the same species as the sperm.

Less Invasive Reproduction Modality

Disclosed herein are methods to identify a sperm suitable for a less invasive reproduction modality or conversely suitability of a less invasive reproduction modality for a given sperm sample. Less invasive reproduction modality comprises providing the sperm evaluated by the methods disclosed herein with an egg in vivo for in vivo fertilization. In vivo access can be provided by, for example, by natural conception. In vivo access can be provided in the reproductive tract of a female subject of the same species as the sperm. In vivo fertilization can be done by artificial insemination of sperm, for example, by intracervical insemination or intrauterine insemination. Standard artificial insemination and intrauterine insemination, and other methods are well known to those of skill in the art. In some embodiments, the sperm is provided access to an egg in the reproductive tract of a female subject by intrauterine insemination of the said sperm to promote fertilization of the egg. In some embodiments, prior to providing access to an egg; a sperm from a mammalian donor or a given sperm sample (e.g., a mammalian donor or a given sperm sample whose sperm has been evaluated to be of higher quality or suitable for a less invasive reproduction modality) is incubated under energy depletion condition to potentiate the sperm. In some embodiments, the potentiated sperm is further provided an effective amount of a first energy source and optionally a second energy source prior to providing access to an egg. In other embodiments, the sperm can be provided the second energy source and access to an egg in vivo by intrauterine insemination of a mammalian sperm which has been incubated under energy depletion conditions and provided the first energy source in vitro. In other embodiments, the sperm can be provided the first and second energy source in vivo by intrauterine insemination of a sperm which has been incubated under energy depleting conditions in vitro. The sperm that is injected, may be used as held in suitable liquids. Liquid used for this purpose may be those liquids generally used as a medium for artificial insemination.

More Invasive Reproduction Modality

Provided herein are methods to identify the suitability of a sperm for more invasive reproduction modality e.g., ART, and in particular IVF or conversely suitability of a more invasive reproduction modality for a given sperm sample. Other suitable ART techniques to which the present disclosure is applicable include, but are not limited to, gamete intrafallopian transfer (GIFT), zygote intrafallopian transfer (ZIFT), blastocyst transfer (BT), intracytoplasmic sperm injection (ICSI), gamete, embryo and cell cryopreservation, in vitro preparation of embryos for embryo biopsy and other forms of embryo micromanipulation including formation of embryos by nuclear transfer and production transgenic lines and genetically modified lines.

In some embodiments, the reproduction methods disclosed herein comprise more invasive reproduction modality. The more invasive reproduction modality comprises providing a sperm with access to an egg in vitro for in vitro fertilization, such as for example, by microinjection, including intracytoplasmic sperm injection (ICSI), and other methods well known to those in the art. Typically, in IVF, after fertilization, the cells are grown to the blastocyst stage and then implanted. In some embodiments, prior to providing access to an egg; a sperm from a mammalian donor or a given sperm sample (e.g., a mammalian donor or a given sperm sample whose sperm has been evaluated to be of higher quality or suitable for a more invasive reproduction modality) is incubated under energy depletion condition to potentiate the sperm. In some embodiments, the potentiated sperm is further provided an effective amount of a first energy source and optionally a second energy source prior to providing access to an egg.

Providing the sperm access in vitro to the egg may be carried out in an appropriate medium. The medium used for this purpose can be a medium generally used as a medium for in vitro fertilization, for example, HTF medium. Temperature conditions for providing access may be a general temperature to be used in vitro fertilization, for example, can be an average body or a temperature close thereto of the mammal. Time for providing access may be any time that is generally required in vitro fertilization, but not particularly limited, and preferably from 6 to 24 hours. In vitro fertilization rate can be determined by incubating one or more sperms with matured oocytes for about 24 hr. Oocytes can then be stained with a 1% aceto-orcein stain to determine the percent fertilized, or left in culture to divide and the number of embryos formed are counted. Oocytes can be matured in vitro in M199 media with 50 μg luteinizing hormone/ml (Brackett and Zuelke, Theriogenology 39:43, 1993)

Starvation

In some embodiments, a method described herein comprises an energy depletion step. In some embodiments one or more of glycolysis, gluconeogenesis, Kreb's cycle, or oxidative phosphorylation are inhibited in the energy depletion and, in particular embodiments, the energy depletion includes glycolytic energy depletion. Exemplary conditions of glycolytic energy depletion include removing substantially all of glycolytically-liable sugar, such as glucose (other embodiments can include, mannose, fructose, dextrose, sucrose, and combinations thereof, including combinations with glucose), in the sperm's medium or reducing the concentration of glycolytically-liable sugar, or using inhibitors of glycolysis, gluconeogenesis, or importers of glycolytically-liable sugars. As glucose is a primary energy source of sperm, in preferred embodiments, the energy depletion is glucose energy depletion (including starvation), which further entails depletion (including starvation) of gluconeogenesis substrates (including, e.g., pyruvate or, in some embodiments lactate, which can be converted to pyruvate by lactate dehydrogenase), and Kreb's cycle substrates (acetyl CoA, citrate, isocitrate, alpha-ketoglutarate, succinyl-CoA, succinate, fumarate, malate, and oxaloacetate).

In some embodiments, the energy depletion comprises a low glucose concentration, e.g., less than about: 0.5, 0.4, 0.3, 0.2, 0.1, 0.09, 0.08, 0.07, 0.06, 0.05, 0.04, 0.03, mM glucose, or less, such as less than about: 0.02 or 0.01 mM, e.g., less than about 0.01 mM. In some embodiments the energy depletion means a substantially glucose-free condition. The invention provides methods entailing staged provision of effective amounts of first and second energy sources and the skilled artisan will appreciate that in some embodiments encompassed within the invention, sub-effective amounts of a glycolytic energy source are an energy depletion and, for example, the foregoing low glucose concentrations can be employed in such embodiments as an energy depletion.

In some embodiments, the energy depletion comprises a low pyruvate concentration, e.g., less than about: 0.15, 0.10, 0.09, 0.08, 0.07, 0.06, 0.05, 0.04, 0.03, 0.02, 0.01, 0.005, 0.003, 0.002 mM, or less. In some embodiments the energy depletion means a substantially pyruvate-free condition. As noted above and exemplified with glucose for a glycolytic energy source, the skilled artisan will also appreciate that in some embodiments encompassed within the invention sub-effective amounts of a gluconeogenesis substrate are an energy depletion and, for example, the foregoing low pyruvate concentrations can be employed in such embodiments as an energy depletion

In some particular embodiments, the energy depletion comprises a condition substantially free of carbon sources, such as low glucose concentration and low pyruvate concentration, e.g., a substantially glucose-free and substantially pyruvate-free condition.

In some embodiments, the energy depletion is for at least about: 10, 20, 30, 40, 50, 60 minutes, e.g., at least about: 30, 40, 45, 50, 55, 60, 90, 120, 150, or 180 minutes or 1, 1.5, 2, 2.5, 3, 3.5, 4, 4.5, 5, 5.5, 6, 6.5, 7, 7.5, 8, 8.5, 9, 9.5, or 10 hours.

Energy depletion consonant with the invention potentiates the sperm. In some embodiments, sperm are potentiated using a suitable induction, e.g., removing or reversing the energy depletion and, e.g., incubating the sperm in capacitation conditions or staged energy reintroduction. In some embodiments, sperm rapidly recover motility, such as one or more of: an increased proportion of hyperactivated, intermediate, or progressive motility sperm (or an increased proportion of a combination of two (such as hyperactivated and intermediate) or all three), and/or increased curvilinear velocities.

Energy Reintroduction

Following energy depletion sufficient to potentiate the sperm, in some embodiments, an effective amount of a first energy source and optionally an effective amount of a second energy source is provided to the potentiated sperm. In some embodiments, the first energy source and the second energy source are provided concurrently. In some embodiments, the first energy source and the second energy source are provided sequentially. In some embodiments, the time between providing an effective amount of a first energy source after potentiating the sperm and providing an effective amount of a second energy source is at least about: 1, 2, 3, 4, 5, 10, 15, 20, 25, 30, 35, 40, 45, 50, 55, or 60 minutes, e.g., at least 5, 6, 7, 8, 9, 10, 11, 12, 13, 14, 15, 16, 17, 18, 19, or 20 minutes, e.g., at least between about: 5-15 minutes. In some embodiments, the time between providing an effective amount of a first energy source after potentiating the sperm and providing an effective amount of a second energy source is longer, such as at least 2, 3, 4, or 5 hours, or more. In other embodiments, the first and second energy source are provided substantially concurrently, e.g., in a single solution or bolus.

In some embodiments, the first energy source is selected from a glycolytic energy source, or a gluconeogenesis substrate. In some embodiments, the second energy source is selected from a glycolytic energy source or a gluconeogenesis substrate, wherein the selected second energy source is not the same as the first energy source. In some embodiments, the gluconeogenesis substrate is pyruvate, e.g., at a concentration of about: 0.15-0.66 mM, e.g., about: 0.20-0.50 mM, such as about: 0.25-0.40 mM, or about: 0.30 mM. The forgoing concentrations are exemplary effective amounts of a gluconeogenesis substrate, for example, when provided as either a first or second energy source in the methods provided by the invention. The skilled artisan will recognize other effective amounts of gluconeogenesis substrates by virtue of their ability to increase sperm function consonant with the teachings of the invention. In some embodiments, the first energy source is a gluconeogenesis substrate, such as pyruvate. In some embodiments, the second energy source is a gluconeogenesis substrate, such as pyruvate.

In some embodiments, the glycolytic energy source is glucose, e.g., at a concentration of about: 0.6 mM-10.0 mM, 1.0-7.0 mM, 2.5-7.0 mM, 3.5-6.5 mM or 5 mM, e.g., at least about: 1, 2, 3, or 4 mM. The forgoing concentrations are exemplary effective amounts of a glycolytic energy source, for example, when provided as either a first or second energy source in the methods provided by the invention. The skilled artisan will recognize other effective amounts of glycolytic energy sources by virtue of their ability to increase sperm function consonant with the teachings of the invention. In some embodiments, the first energy source is a glycolytic energy source, such as glucose. In some embodiments the second energy source is a glycolytic energy source, such as glucose. In some embodiments, the first energy source is a glycolytic energy source, such as glucose, while the second energy source is a gluconeogenesis substrate, such as pyruvate.

An additional condition regulated in some embodiments of the methods provided by the invention is the osmolarity (mOsm) or osmolality (mOsm/kg). In some embodiments, the method is performed at an osmolarity (or osmolality) ranging from between about: 200-280 mOsm (mOsm/kg), e.g., between about: 220-260, 225-255, 230-250 mOsm (mOsm/kg) during energy depletion, optionally, wherein upon addition of the first or second energy source, the osmolarity (or osmolality) is increased to at least about: 270, 275, 280, 285, 290, or 295 mOsm (mOsm/kg).

Gluconeogenesis substrate suitable for use in the methods of the present disclosure include, but are not limited to, pyruvate, lactate, succinate, citrate, fumarate, malate, aspartate, glycerol, acetyl CoA, isocitrate, alpha-ketoglutarate, succinyl-CoA, oxaloacetate; or a physiologically acceptable derivative, salt, ester, polymer or alpha-keto analogue of the gluconeogenesis substrate. Any gluconeogenic amino acid, or a physiologically acceptable derivative, salt, ester, or polymer, or alpha-keto analogue thereof is also suitable as a gluconeogenesis substrate. Non-limiting examples of gluconeogenic amino acids include alanine, arginine, asparagine, cystine, glutamine, glycine, histidine, hydroxyproline, methionine, proline, serine, threonine and valine. Non-limiting examples of pharmaceutically acceptable salts of pyruvate are lithium pyruvate, sodium pyruvate, potassium pyruvate, magnesium pyruvate, calcium pyruvate, and zinc pyruvate. In some embodiments, the pyruvate is sodium pyruvate. Non-limiting examples of salts of lactate include sodium lactate, potassium lactate, magnesium lactate, calcium lactate, zinc lactate, and manganese lactate. The gluconeogenesis substrate of the methods disclosed herein can be any one of the gluconeogenesis substrates listed above.

Glycolytic energy source suitable for use in the methods of the present disclosure include but are not limited to carbon sources for glycolysis. Non-limiting examples of glycolytic energy source useful in the methods disclosed herein include monosaccharides (such as fructose, glucose, galactose and mannose) and disaccharides (sucrose, lactose, maltose, and trehalose), as well as polysaccharides, galactose, oligosaccharides, polymers thereof.

In some embodiments of the methods provided by the invention, additional components are provided to the sperm. For example, other components upstream and downstream of glycolysis such as NADH, NAD⁺, citrate, AMP, ADP, or a combination thereof are added in combination with at least the first energy source or the second energy source.

Some embodiments of the methods provided by the invention include assessment of the sperm. For example, in some embodiments, the methods include one or more quantitative assessments of sperm motility, e.g., by CASA, and/or measuring sperm quality, such as DNA fragmentation (e.g., by TUNEL), lipid peroxidation, reactive oxygen species, or a combination thereof.

The methods provided by the invention achieve increased sperm function. In some embodiments, relative to a suitable control sperm. In some embodiments the suitable control is sperm in standard capacitation medium (C-HTF), without a starvation step, while in some embodiments, the suitable control is sperm in standard capacitation medium (C-HTF) following a three hour starvation—e.g., starvation and reintroduction of effective amounts of energy sources without staging reintroduction of the energy sources. In some embodiments, a suitable control sperm is a non-capacitated sperm.

Mammalian Sperm

The methods disclosed herein comprise evaluating a sperm and/or identifying a suitable reproduction modality for a sperm. As used herein, the sperm can be from a vertebrate, preferably a mammal. Accordingly, the sperm of the present disclosure can be a mammalian sperm. The sperm can be from a mammalian donor.

Mammals include, without limitation, humans, primates, rodents, wild or domesticated animals, including feral animals, farm animals, sport animals, and pets. Rodents include, for example, mice, rats, woodchucks, ferrets, rabbits and hamsters. Domestic and game animals include, for example, cows, horses, pigs, deer, bison, buffalo, feline species, e.g., domestic cat, and canine species, e.g., dog, fox, wolf, avian species, e.g., chicken, emu, ostrich, and fish, e.g., trout, catfish and salmon. The mammalian sperm can be from a non-human mammal including, an ungulate, such as an even-toed ungulate (e.g., pigs, peccaries, hippopotamuses, camels, llamas, chevrotains (mouse deer), deer, giraffes, pronghorn, antelopes, goat-antelopes (which include sheep, goats and others), or cattle) or an odd-toed ungulate (e.g., horse, tapirs, and rhinoceroses), a non-human primate (e.g., a monkey, chimpanzees, cynomologous monkeys, spider monkeys, and macaques, e.g., Rhesus.), a Canidae (e.g., a dog) or a cat. The mammalian donor of sperm can be from a member of the Laurasiatheria superorder. The Laurasiatheria superorder can include a group of mammals as described in Waddell et al., Towards Resolving the Interordinal Relationships of Placental Mammals. Systematic Biology 48 (1): 1-5 (1999). The Members of the Laurasiatheria superorder can include Eulipotyphla (hedgehogs, shrews, and moles), Perissodactyla (rhinoceroses, horses, and tapirs), Carnivora (carnivores), Cetartiodactyla (artiodactyls and cetaceans), Chiroptera (bats), and Pholidota (pangolins). A member of Laurasiatheria superorder can be an ungulate, e.g., an odd-toed ungulate or even-toed ungulate. An ungulate can be a pig. The mammalian donor can be from a member of Carnivora, such as a cat, or a dog. In some embodiments, the mammalian donor is a human, non-human primate, porcine, bovine, equine, ovine, canine, feline, or murine sperm. In some embodiments, the mammalian donor is a human donor. In some embodiments, the sperm is a human sperm.

In some embodiments, the mammalian sperm is from a healthy male mammal. In some embodiments, the sperm is from a male suffering from sperm dysfunction, for example, low sperm count, reduced motility of sperm, and abnormal morphology of sperm. In some embodiments, the mammalian donor can be from a subfertile male or an oligospermic male. The mammalian donor can be a male suffering from, for example, oligospermia, Teratozoospermia, Asthenozoospermia, or Oligoasthenoteratozoospermia. Oligospermia refers to a condition characterized by sperm concentration of <20 million/ml. Asthenozoospermia refers to a condition characterized by reduced sperm motility. Teratozoospermia refers to a condition characterized by presence of sperm with abnormal morphology. Oligoasthenoteratozoospermia refers to a condition that includes oligozoospermia (low number of sperm), asthenozoospermia (poor sperm movement), and teratozoospermia (abnormal sperm shape). In some embodiments, the sperm is obtained from a subfertile male or an oligospermic male, e.g., having a sperm count below about: 20, 19, 18, 17, 16, 15, 14, 13, 12, 11, 10, 9, 8, 7, 6, 5, 4, 3, 2 million sperm per milliliter, e.g., less than 15 million sperm per milliliter. In some embodiments, the sperm is obtained from a subfertile male comprising a lower curvilinear velocity of sperm, for example, by at least 5%, 10%, 20%, 30%, 40%, 50%, 60%, 70%, 80%, or more than the range of normal for a fertile male.

In some embodiments, the sperm are enriched (or isolated) from semen prior to energy depletion. Any method of sperm enrichment or isolation can be used consonant with the invention, including density gradient centrifugation, swim up, microfluidics, or a combination thereof.

Sperm may be used in the methods provided by the invention either fresh or from a preserved stock. For example, in some embodiments, prior to treatment, the sperm are recovered from cryogenic storage. In other embodiments, prior to treatment, the sperm are recovered from non-cryogenic storage.

Different quantities of sperm can be used in the methods provided by the invention, including fractions of a single ejaculate or a whole ejaculate. In some embodiments, the sperm are pooled from two or more ejaculates (e.g., 2, 3, 4, 5, 6, or more ejaculates).

Methods of Obtaining Sperm Sample

Various methods of collection of viable sperm are known. Such methods include, for example, masturbation into sterile containers, the gloved-hand method, use of an artificial vagina, and electro-ejaculation. Animal semen can be collected by using artificial vagina, electro-ejaculator, or by massaging the ampule of the animal by hand. It can also be directly collected from any section of the male reproductive tract including testicular sperm, and sperm obtained from caput, corpus or cauda epididymis using different methodologies such as puncture of the testis or epididymis using surgical procedures or removing the testis or epididymis and collecting the sperm in surrounding media. The sperm are preferably collected or quickly transferred into an insulated container to avoid a rapid temperature change from physiological temperatures (typically about 35° C. to about 39° C.). The ejaculate typically contains about 0.01 to 15 billion sperm per milliliter, depending upon the species and particular animal. The number may be reduced if obtained from a subfertile male or male suffering from sperm dysfunction.

The sperm may be freshly collected sample from a source animal (e.g., a mammal), or can be previously thawed or cryopreserved sample. At the time of collection, or subsequently, the collected sperm may be combined with any of a number of various buffers that are compatible with sperm, such as TCA, HEPES, PBS, or any of the other buffers disclosed in U.S. Patent Application Publication No. US 2005/0003472, the content of which is hereby incorporated herein by reference. For example, a bovine semen sample typically containing about 0.5 to about 10 billion sperm cells per milliliter may be collected directly from the source mammal into a vessel containing a buffer to form a sperm suspension. The sperm suspension may also contain a range of other additives to maintain sperm viability. Exemplary additives include protein sources, antibiotics, growth factors, and compositions that regulate oxidation/reduction reactions intracellularly and/or extracellularly. Examples of each of these additives are well known in the art, as demonstrated in the disclosure of, for example, U.S. Application Ser. Nos. 60/557,407 and 11/092,313, the content of each of which is hereby incorporated herein by reference. Alternatively, the semen sample may be collected into an empty container and then subsequently contacted with a buffer within several minutes to hours after collection to form the sperm suspension. In some embodiments, the sperm cells can be collected directly into a container containing energy depletion medium (e.g., HTF medium devoid of glucose, pyruvate and/or lactate) for incubation under energy depletion. In some embodiments, the sperm cells can be collected in an empty container and subsequently incubated under energy depleting conditions.

In some embodiments, sperm collection comprises washing sperm cells prior to carrying out the methods disclosed herein. Generally, washing involves centrifuging a sample of semen or thawed sperm through a diluting wash media, which allows collection of a sperm-rich pellet. After a sperm wash process, or in place of it, a specific procedure for the isolation of the motile sperm from a sample can be done.

In some embodiments, the sperms are isolated from semen prior to use in methods disclosed herein. In some embodiments, sperm with increased function can be further enriched, (for example, enriching sperm with increased motility), from sperm prepared according to methods disclosed herein. Generally, sperm are isolated or enriched by allowing the motile sperm to swim away from the dead sperm, non motile sperm and debris (sperm swim-up), by centrifuging the sperm through a density gradient, or by passing the sperm through a column that binds the dead sperm and debris. Isolating (or enriching) the spermatozoa from semen is performed by a method selected from the wash and spin method, the sedimentation method, the direct swim-up method, the pellet and swim-up method, and the buoyant density gradient method. These methods are well known in the art. They are traditionally used in assisted reproduction techniques and described in detail in “A textbook of In vitro Fertilization and Assisted Reproduction, The Bourn Hall guide to clinical and laboratory practice, editor: Peter R. Brinsden, The Parthenon Publishing Group” (1999). In some embodiments, the sperm prepared by the methods disclosed herein can be further enriched for motile sperms by isolation procedures such as the sedimentation method, the direct swim-up method, the pellet and swim-up method, and the buoyant density gradient method.

The direct swim-up method implies self-selection of motile sperms, essentially comprising layering an aliquot of medium on top of a semen sample or a preparation of sperm disclosed herein and allowing it to stand at room temperature for a certain period of time. The motile sperm cells will migrate into the top layer (medium), from which they can be recovered. The method may also include centrifugation step(s). The advantage of “swim-up” selected spermatozoa is that the motile cells present in the sample are isolated and concentrated and that the proportion of morphologically normal sperm is increased.

The method may be varied and combined with further isolation/separation techniques, depending on the amount of motile cells in the sample. For example, the swim-up procedure may be performed through the layering of 1 ml of medium containing albumin on 1 ml of underlying seminal liquid in a test tube. After one hour of incubation at 37° C. in the air or in 5% CO2 the upper phase of the medium to which the spermatozoa with better motility characteristics have migrated is collected. This technique may also comprise or be combined with a centrifugation step, for example centrifugation on Percoll gradients. In typical applications, a sperm containing solution is layered over a gradient material, preferably Percoll at 30-90% mixed with 0.05% pectin, and then subjected to centrifugation to collect sperm enriched for improved function. The separated, isolated or enriched spermatozoa are then used in methods disclosed herein or may be cryopreserved before being further processed, for example. In case of the preparation of sperms prepared by methods herein, they can be used for IVF, ICSI or artificial insemination following enrichment steps or may be cryo-preserved for later use, for example. Accordingly, for any of these isolation, or enrichment methods, the sample may be semen, partially purified sperm, purified sperm, or sperm with increased function prepared by methods herein. In some embodiments, the percentage of motile cells is increased by at least 10%, at least 20%, at least 50%, at least 75%, at least 80%, or about 100% after isolating or enriching the sperm using isolation methods, such as direct swim up, the pellet and swim-up method, and the buoyant density gradient method compared to untreated semen sample or unenriched sperm preparation.

In some embodiments, after isolation, enrichment and washing, the sperm pellet can be resuspended in a medium suitable for further processing, including preservation medium, HTF medium for culturing, medium for energy depleting conditions (e.g., HTF devoid of glucose, lactate and/or pyruvate). As it relates to sperm with increased function prepared by methods disclosed herein, the sperm preparation can be resuspended in preservation medium, HTF medium for culturing, medium for insemination, assays of fertilization potential as described herein, in vitro fertilization, freezing, intrauterine insemination, cervical cap insemination, and the like. The sperm may be added to medium or the medium can be added to the sperm. The medium can be balanced salt solution which may contain zwitterionic buffers, such as TES, HEPES, PIPES, or other buffers, such as sodium bicarbonate. In general, the medium for diluting sperm or culturing sperm, oocytes, embryos or embryonic stem cells is a balanced salt solution, such as M199, Synthetic Oviduct Fluid, PBS, BO, Test-yolk, Tyrode's, HBSS, Ham's F10, HTF, Menezo's B2, Menezo's B3, Ham's F12, DMEM, TALP, Earle's Buffered Salts, CZB, KSOM, BWW Medium, and emCare Media (PETS, Canton, Tex.). In some embodiments, TALP or HTF is used for sperm culture medium, and CZB is used for embryo culture medium. The sperm, or embryo of the present disclosure can be preserved in a cryogenic medium comprising a cryoprotectant.

Suitable Control

A suitable control can be sperm incubated under control conditions, i.e., in a control buffer. The control condition can comprise, for example, incubating sperm under standard capacitation conditions. HTF comprises a sodium bicarbonate buffering system and may be utilized for uses requiring a carbon dioxide atmosphere during incubation. Modified HTF comprises a combined sodium bicarbonate and HEPES ([4-2(2-hydroxyethyl)-1-piperazineethanesulfonic acid]) buffer. Suitable examples of HTF medium or modified HTF medium include those that are commercially available from Irvine Scientific, Santa Ana, Calif. In some embodiments, the incubating in energy depletion conditions can be incubating the HTF medium from which glucose, lactate and pyruvate has been omitted. The sperm may be incubated for a period sufficient to provide a measurable change in sperm function (e.g., the motility) or other characteristics of the sperm; in specific embodiments of the method, incubation in control condition or standard capacitation conditions is from 1 minute to 24 hours, 15 minutes to 3 hours, 30 minutes to 1.5 hours, about 1 hour, or any subrange or subvalue thereof. In some embodiments, a suitable control is sperm which is incubated in energy depletion conditions followed by treatment with a first energy source (e.g., selected from a gluconeogenesis substrate, or a glycolytic substrate) or a second energy source (e.g., selected from a gluconeogenesis substrate, or a glycolytic substrate but not same as first energy source) independently. In some embodiments, a suitable control is sperm which is incubated in energy depletion conditions followed by treatment with a gluconeogenesis substrate, or a glycolytic substrate independently. In some embodiments, a suitable control is a sperm which is incubated in energy depletion conditions followed by treatment with a first energy source and a second energy source simultaneously. In some embodiments, a suitable control is a sperm which is incubated in energy depletion conditions followed by treatment with a gluconeogenesis substrate and a glycolytic substrate simultaneously. In some embodiments, a suitable control is an untreated sperm, e.g., a sperm not subjected to energy depletion condition. In some embodiments, a suitable control is an untreated sperm, e.g., a sperm not subjected to capacitation conditions. It is understood that a suitable control can be at least one sperm or a population of sperm, for example, a sperm preparation, or a sperm suspension. Similarly, controls may be substantially contemporaneous parallel measurements, e.g., in fractions of a single sample, or a donor-matched sample, or numerical thresholds previously calculated for the subject, for similarly situated subjects, or other previously determined reference values.

It will be understood that in the methods provided by the invention that one control—e.g., sperm treated under standard capacitation medium or untreated controls—is, in some embodiments, sufficient to calculate, for example, a ratio of the percentage of hyperactivated and/or intermediate motility sperm under treatment and control conditions. In other embodiments, comparisons can be made using two or more (e.g., 3, 4, 5, or more) controls, for example using some function of, for example, the percentage of hyperactivated and intermediate motility sperm under the different controls, relative to the treatment (starve-refeed). The function may be a ratio of the treatment to, for example, an average of all of the different controls, the top control (or top 1, 2, 3, for example), the bottom control (or bottom 1, 2, 3, for example), or middle controls (e.g., omitting the top and bottom 1, 2, or 3 controls). In addition to ratios, other functions of the activity (e.g., as measured by the percentage of hyperactive and intermediate motility sperm) of treatment and controls, including differences, threshold classifiers, et cetera.

Sperm Preparation

In some embodiments, the disclosure provides sperm preparations, such as preparations of sperm identified to be of higher sperm quality by methods disclosed herein, or preparation of sperm evaluated to be suitable for a selected reproduction modality using methods disclosed herein. The preparation of sperm can comprise activated (e.g., sperm having been starved following introduction of an effective amount of a first and optionally a second energy source, enriched for hyperactivated and intermediate sperm), partially activated sperm (sperm having been starved and contacted with an effective amount of only a first energy source), or potentiated sperm. These are collectively “sperm preparations” provided by the disclosure or “preparations provided by the disclosure.” In some embodiments, the disclosure provides preparations of hyperactivated sperm comprising at least 5% hyperactivated sperm, e.g., at least about: 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, 20.0%, or more hyperactivated sperm, e.g., between about: 5-20, 8.5-20, 10-20, or 12.5-20%. In some embodiments, the preparation also contains at least about: 5.5, 6.0, 6.5, 7.0, 7.5, 8.0, 8.5, 9.0, 9.5, 10.0, 12, 14, 15, 16, 18, 20, 22, 24, 25, 26, 28, or 30% intermediate motility sperm, e.g., between about: 20.5-30%, 22.5-30%. Thus, in some embodiments, the percentage sum of hyperactivated and intermediate motility sperm is at least: 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, 20.0, 21.0, 21.5, 22.0, 22.5, 23.0, 23.5, 24.0, 24.5, 25, 26, 27, 28, 29, 30, 35, 40, 41, 42, 43, 44, 45, 46, 47, 48, 49, 50%, or more, e.g., between about: 10-50, 30.5-50, 32.5-50. As the skilled artisan will appreciate, sperm may be separated based on hyperactivation (and/or intermediate) phenotype, but in some embodiments, the foregoing percentages are based on preparations that have not been activated and then sorted based on hyperactivation (however, in some embodiments, sperm preparations may have been pre-processed, e.g., to separate or otherwise enrich sperm from other seminal components, including certain irregular sperm). In some embodiments, the hyperactivated (or intermediate motility, or hyperactivated and intermediate motility) sperm in the preparation have 10, 15, 20, 25, 30, 35, 40, 45, 50%, or more (e.g., 2, 3, 4, 5, 6, 7, 8, 9, 10-fold, or more) reduction in intracellular RNA concentration (such as small non-coding RNAs, including microRNA), relative to a suitable control. In some embodiments sperm in a preparation provided by the invention are characterized (as assessed by either bulk average metrics or percentages in categories) by altered sperm head morphology, increased tail movement (e.g., amplitude), or a combination thereof.

In some embodiments, the percentage of hyperactivated sperm and intermediate motility sperm in the preparation is higher relative to the percentage of hyperactivated sperm and intermediate motility sperm in a suitable control by a factor greater than 1, e.g., greater than about: 1.05, 1.1, 1.2, 1.3, 1.35, 1.4, 1.45, 1.5, 1.55, 1.6, 1.65, 1.7, 1.75, 1.8, 1.85, 1.9, 1.95, or more, e.g., greater than about: 2, 3, 4, 5, 6, 7, 8, 9, or, 10.

In some embodiments, the invention provides preparations of sperm prepared by enriching sperm from semen of a male subject, such as a normospermic male, sub fertile male, or oligospermic male, e.g., a subfertile (including oligospermic) male, whose sperm was previously evaluated to be high quality sperm or determined to be suitable for a reproduction modality.

For any of the preparations provided by the invention, sperm can be from any male subject, such as a mammal, and in some embodiments, a human. In some embodiments, the human is a normospermic male, or in other embodiments, the male is an oligospermic or subfertile (e.g., low sperm motility) subject.

Fertilization Uses

Provided herein are methods of evaluating sperm quality and methods to determine suitability of a sperm for a reproductive modality. These methods disclosed herein are generally applicable to many species, including human, bovine, canine, equine, porcine, ovine, avian, rodent and others. The methods are useful whenever successful fertilization is desired, or to inform selection of a suitable reproduction modality for a sperm. The present methods have particular use in animals and humans that have a fertilization dysfunction in order to increase the likelihood of conception. Such dysfunctions include low sperm count, reduced motility of sperm, and abnormal morphology of sperm. Accordingly, the methods disclosed herein can be useful for identifying samples with higher sperm quality in infertility clinics or to identify suitable reproduction modality for a sample, prior to their use in vitro fertilization, intrauterine insemination or ICSI. The methods described herein can be used to predict and improve likelihood of success in artificial insemination, IVF or ICSI in exotic species and/or endangered species. As such the methods can find use for evaluating sperm quality for animals maintained captive in a zoo, and in conservation programs aiming to improve rate of successful fertilization attempts in animals that are close to extinction in the wild. For example, the methods of the present disclosure can be used to evaluate sperm quality prior to its use in a reproduction modality in order to improve fertilization and pregnancy rate in animal husbandry, for species of agricultural value, and in species bred for conservation purposes.

In addition, the methods and compositions of the present invention are useful in artificial insemination procedures, e.g., in commercial breeding. The method can be carried out with sperm from domesticated animals, especially livestock, as well as with sperm from wild animals (e.g., endangered species). For example, as disclosed herein, embodiments of the methods and compositions of the disclosure find application in bovine reproduction. The methods and preparation can be useful for artificial insemination in the livestock production industry where it is desirable to select sperm with higher sperm quality to influence the outcome towards offspring having one or more preferred characteristics or traits by introducing specific genetically-determined traits into the livestock, e.g., offspring of a particular gender, offspring with enhanced milk production, offspring for quality meat production. Use of the prognostic methods described herein prior to use of sperm for fertilization will result in improved pregnancy rates. Mammalian sperm are frequently damaged by freezing and thawing and results in lower fertility. By improving the likelihood of successful fertilization attempt, sperm evaluated by methods disclosed herein when used for insemination may promote a higher pregnancy rate per estrus cycle, reducing the number of cycles required to ensure conception and hence reducing the overall cost of artificial insemination.

Semen from animals with highly desirable traits could be used to inseminate more females because fewer cycles would be needed to ensure conception in any one female. For such applications, the semen is obtained from a male with desired characteristics. In order to influence gender outcome of the resulting offspring, the sperm preparation can be sorted into X- and Y chromosome bearing cells, and/or enriched for sperm with one or more increased sperm function disclosed herein. The sperm may be sorted by commonly used methods, for example, as described in Johnson et al. (U.S. Pat. No. 5,135,759) using a flow cytometer/cell sorter into X and Y chromosome-bearing sperm enriched populations. The sperm prepared by the methods disclosed herein can be sorted the into a population comprising a certain percent X chromosome bearing or Y chromosome bearing sperm cells. For example, the spermatozoa of one of the populations may comprise at least about 65% X chromosome bearing or Y chromosome bearing sperm cells, at least about 70% X chromosome bearing or Y chromosome bearing sperm cells, at least about 75% X chromosome bearing or Y chromosome bearing sperm cells, at least about 80% X chromosome bearing or Y chromosome bearing sperm cells, at least about 85% X chromosome bearing or Y chromosome bearing sperm cells, at least about 90% X chromosome bearing or Y chromosome bearing sperm cells, or even at least about 95% X chromosome bearing or Y chromosome bearing sperm cells. In some embodiments, the sorting can be done prior to prior to performing the methods disclosed herein. In some embodiments, the sorting can be done after performing the methods disclosed herein, but prior to providing the sperm with increased function with access to an egg for fertilization as in IVF, ICSI, or AI.

The methods and preparations provided by the invention can be used in assisted fertilization, such as IUI, IVF, including by ICSI (intracytoplasmic sperm injection). The methods disclosed herein are useful for informing selection of a suitable reproduction modality for a given sperm sample, or conversely identifying the suitability of a sperm sample for use with a selected reproduction modality. In some embodiments, any of the reproduction methods provided by the invention can include the step of providing the sperm to a female reproductive tract, optionally wherein the effective amount of a second energy source is provided in the female reproductive tract. In some embodiments, a sperm can be provided access to an egg for a time sufficient to fertilize the egg, which egg may be ex vivo (e.g., IVF, including ICSI) or, in some embodiments, in a female reproductive tract. Such methods, in some embodiments, entail a subsequent implantation of the fertilized egg in a female carrier.

Articles of Manufacture

In some embodiments, the invention also provides articles of manufacture and kits, e.g., suitable for performing any of the methods provided by the invention. Provided herein are kits for screening sperm samples with higher sperm quality. The kits disclosed herein are useful to identify a reproduction modality suitable for a potential donor's sperm. The kits disclosed herein are useful to identify whether a selected reproduction modality is suitable for a donor's sperm. For example, in some embodiments, the disclosure provides articles of manufacture comprising a sperm potentiating solution that, upon contact with sperm, induces energy depletion; a first solution providing a first energy source (such as an effective amount of a glycolytic energy source or an effective amount of a gluconeogenesis substrate), and optionally a second energy source, or optionally a second solution providing an effective amount of a second energy source. In some embodiments, the articles of manufacture further include a means for isolating or purifying sperm, such as a sperm isolating matrix, microfluidic device, etc. In some embodiments, the sperm isolating matrix is silanized silica, optionally wherein the silanized silica is in media substantially free of any glycolytic energy source or gluconeogenesis substrate. In some embodiments, the kit comprises instructions for carrying out the methods disclosed herein. The kit can also include a washing medium, a preservation medium, culture medium (e.g., HTF), a diluent, and the like. The kits can further contain adjuvants, reagents, and buffers necessary.

The kits can also include a carrier, package, or container that is compartmentalized to receive one or more containers such as vials, tubes, and the like, each of the container(s) comprising one of the separate elements, such as the potentiating solution, first solution providing the first energy source, and second solution providing the second energy source to be used in a method described herein. Suitable containers include, for example, bottles, vials, syringes, and test tubes. The containers can be formed from a variety of materials such as glass or plastic. The articles of manufacture provided herein contain packaging materials. Examples of pharmaceutical packaging materials include, but are not limited to, blister packs, bottles, tubes, bags, containers, bottles, and any packaging material suitable for use in methods disclosed herein. A kit typically includes labels listing contents and/or instructions for use, and package inserts with instructions for use. A set of instructions can also be included.

Computer Systems

Various embodiments described herein may be implemented on one or more hardware, software, or a combination of hardware and software on computer systems, e.g., specially programmed computer systems. It should be appreciated that one or more of any type computer system may be used to perform a process or processes for, e.g., evaluating sperm function, e.g., evaluating sperm motility, e.g., using the CASA system. In some embodiments, the system comprises a computer and a microscope. In some embodiments, the system comprises a camera configured to provide microscope data (e.g., as video) to a computer. Further, the system may be located on a single computer or may be distributed among a plurality of computers attached by a communications network. There are many examples of computer systems currently in use. Some examples include, among others, network appliances, personal computers, workstations, mainframes, networked clients, servers, media servers, application servers, database servers, web servers, and virtual servers. Other examples of computer systems may include mobile computing devices, such as cellular phones and personal digital assistants, and network equipment, such as load balancers, routers and switches.

In some embodiments, a general-purpose computer system, software, hardware, or combination thereof according to some embodiments may be specially configured to perform any of the described functions, including but not limited to, acquiring calculation values (e.g., VAP, VSL, VCL, LIN, and ALH), normalizing calculation values against a control, calculating intermediate values, and/or calculating classification value(s) (e.g., classification of sperm motility patterns).

Reporting

Methods described herein can include providing a report, such as, in electronic, web-based, or paper form, to the patient or to another person or entity, e.g., a physician (e.g., a reproductive endocrinologist, a urologist, an obstetrician, or a gynecologist), a hospital, clinic, third-party payor, insurance company, a laboratory, or office. The report can include output from the method, e.g., the identification of a mammalian sperm donor as suitable for a reproductive modality (e.g., a more-invasive modality or a less-invasive modality) or the evaluation of sperm quality of a sperm of a mammalian donor. In some embodiments, a report is generated, such as in paper or electronic form, which identifies the sperm quality or identifies whether a mammalian sperm donor is suitable for a reproductive modality.

The report can also include information on the evaluation of sperm quality and/or sperm function. Such information can include information on a suggested reproductive modality for the donor. The report can include information on the likely effectiveness of a reproductive modality or the advisability of performing a reproductive modality. For example, the report can include information, on the use of a reproductive modality. The report can be delivered, e.g., to an entity described herein, within 7, 14, or 21 days from receipt of the sample by the entity practicing the method.

### EXAMPLES

The present disclosure will be described in greater detail by way of the following specific examples. The following examples are offered for illustrative purposes, and are not intended to limit the invention in any manner. Those of skill in the art will readily recognize a variety of non-critical parameters that can be changed or modified to yield alternative embodiments according to the invention.

### Example 1: Materials and Methods

Media

Media for human sperm capacitation was Human Tubal Fluid (Complete HTF or C-HTF or Standard control) medium, containing 97.8 mM NaCl, 4.7 mM KCl, 2 mM CaCl₂), 0.37 mM KH₂PO₄, 0.2 mM MgSO₄. 7H₂O, 25.1 mM NaHCO₃⁻, 0.33 mM Na-pyruvate, 2.78 mM glucose, lactate 21.4 mM and 5 mg/mL human serum albumin (HSA), 10 μg/mL gentamicin and phenol red 0.0006% at pH 7.4 equilibrated with 5% CO2. For sperm starvation treatment glucose, lactate and pyruvate were omitted from the HTF media above (F-HTF, test media).

Semen Samples

Semen samples were obtained from males seeking treatment for infertility by masturbation into sterile containers. Ejaculates were liquified for up to 6 hours prior to processing for the experiment. Following liquefaction, the volume of the ejaculate was divided equally for processing into F-HTF (test) conditions or C-HTF (control conditions). Semen samples were processed by density-gradient centrifugation to collect viable sperm cells.

Sperm Processing

Density Gradient Preparation Prior Sperm Processing

100% Percoll gradient (Sigma, P-1644) was diluted to 90% with a 10×saline buffer to bring a final concentration of 3 mM KCl, 0.29 mM KH₂PO₄, 80 mM NaCl, 2 mM CaCl₂, 0.4 mM MgCl₂x6H₂O, 20 mM Sodium DL-lactate, 25 mM NaHCO₃ and 10 mM Hepes, to make the lower layer. In the case for the treated sperm (starve sperm), sodium lactate was removed in order to make 90% layer free of nutrients. To make the upper layer (45%), the lower layer (90%) was diluted 1:1 ratio with HTF. The control was diluted with C-HTF and treated with F-HTF.

Density Gradient Centrifugation

Following liquefaction, the entire volume of each ejaculate was equally divided over two different gradient conditions. The test sample was prepared using a 45-90% Percoll (Sigma, P-1644) gradient with the lack energy source (sodium lactate) and F-HTF. The control sample was prepared using 45-90% Percoll (Sigma, P-1644) gradient in the presence energy source (sodium lactate) and C-HTF. Both samples were centrifuged for 20 min at 500×g. Following centrifugation, the supernatant was removed, and the pellet washed with 10 ml media. The test sample was washed in F-HTF and the control sample was washed in C-HTF.

Sperm Starve Stage Rescued

Following density gradient centrifugation process, sperm was incubated in F-HTF or C-HTF media for one hour. Sperm in F-HTF media was divided in three different conditions: 1-) all nutrients were introduced back (glucose, pyruvate and lactate-free), 2-) only glucose (2.5 mM) was introduced back and 3-) only pyruvate (0.33 mM) was introduced back. After one hour, Pyruvate was introduced to sperm primed with glucose alone (2.5 mM) and glucose was introduced back to sperm primed with pyruvate alone (0.33 mM). And the standard control remains in the same C-HTF media for the whole process. Then, 15 minutes later all the conditions Standard control (SC), 1-) Starve rescued (SR), 2-) Starve Primed Glucose (nG) 3-) Starve Primed Pyruvate (nP)) were recorded on the IVOS II CASA system computer-assisted semen analysis (CASA) system (Hamilton Thorne Research, Beverly, Mass.).

Analysis of Sperm Motility

Sperm suspensions of test and control sperm (3 μl) were loaded into one pre-warmed chamber slide (depth, 20 μm) (Leja slide, Spectrum Technologies) and placed on a microscope stage at 37° C. Sperm motility was examined using the IVOS II computer-assisted semen analysis (CASA) system (Hamilton Thorne Research, Beverly, Mass.). One-second tracks were captured using the following settings: 60 frames per second, 60 frames acquired, minimum contrast=80, minimum size=3 pixels, default cell size=6 pixels, default cell intensity=160, slow cells counted as motile, low VAP cutoff=10 μm/s, low VSL cutoff=0 μm/s, minimum intensity gate=0.18, maximum intensity gate=1.21, minimum size gate=0.56 pixels, maximum size gate=2.63 pixels, minimum elongation gate=0 pixels, and maximum elongation gate=99 pixels. Raw data were sorted and analyzed using the CASAnova parameters (Goodson et al., 2018, supra). At least 20 microscopy fields corresponding to a minimum of 500 sperm were analyzed in each experiment.

### Example 2

Purpose

This example demonstrates the classification of responders and non-responders to the starve-refeed protocol.

Materials/Method

Discarded semen samples were collected from 116 anonymous donors seeking fertility treatment and 7 anonymous control donors who were not seeking fertility treatment. Samples were washed and processed using density gradient centrifugation protocol described previously in Example 1.

Each donor sample was split into four equal subsamples, with each subsample subjected to a variation on the capacitation protocol outlined in Example 1. One subsample was subjected to Standard Capacitation conditions (SC). One subsample was subjected to standard Starve-Refeed conditions (SR). The last two were subjected to variations on staged starve-refeed wherein one was subjected to glucose priming followed by pyruvate rescue (nP), the other to pyruvate priming followed by glucose rescue (nG). Sperm motility changes resulting from the four protocols were assessed using computer aided sperm analysis (CASA) following the protocol outlined in Example 1.

Sperm motility for every sperm cell in the field of view was captured using the CEROS II CASA system (Hamilton Thorne Research, Beverly, Mass.). The kinematic parameters for each tracked sperm cell in the field of view were used as input to a custom written support vector machine (SVM) classifier based on CASAnova (Goodson et al. 2017). The classifier assigned each motile sperm in the image field of view to one of five motility classes: Weak, Slow, Progressive, Intermediate or Hyperactivated. Multiple fields of view were assayed until roughly 500 cells per condition were measured.

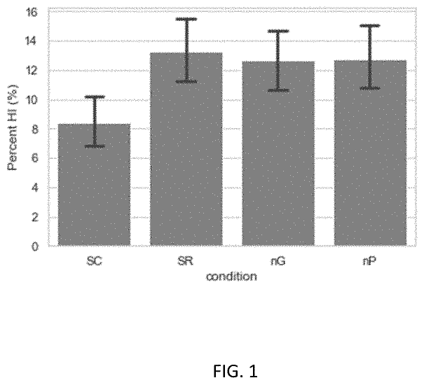

For every donor, the percentage of total cells that were classified as either Hyperactivated or Intermediate (Percent HI) was calculated for each of the four conditions (SC, SR, nC, nP). It was found that all three starve-refeed conditions resulted in an enhancement of Percent HI compared to standard capacitation, as shown in FIG. 1.

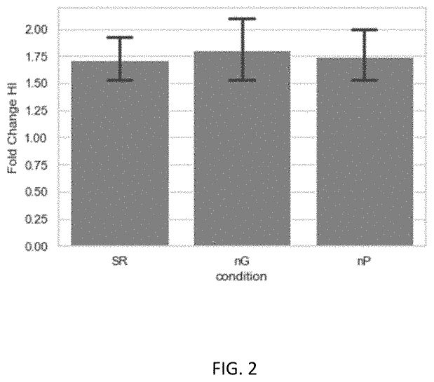

To quantitatively measure the increase in Percent HI of the SR, nG, nP samples over the SC samples the Fold Change of Hyperactivated and Intermediate cells (FC HI), defined as the ratio of the Percent HI of the SR, nP, nG sample to the Percent HI of the SC sample, was computed. It was found that looking at the population level, all three starve-refeed conditions (SR, nG, nP) had a mean fold change higher than one, as shown in FIG. 2, suggesting an enhancement in Percent HI resulting from the SR, nP, nG over the SC treatment.

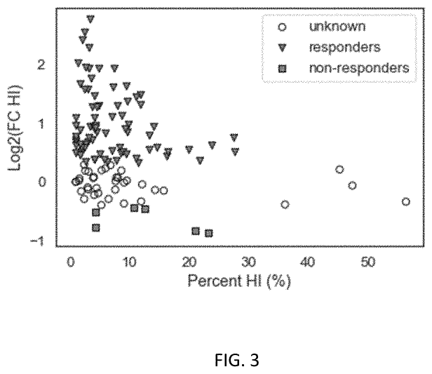

Looking at the individual donor level, it was found that donors exhibited three different responses to the starve-refeed protocol. Each donor sample was classified into one of three classes: responder, non-responder, and unknown depending on its FC HI value. A sample was classified as a responder if its FC HI is larger than a threshold value of 1.25. Alternatively, it was classified as a non-responder if FC HI<0.75. Finally it was classified as unknown if 0.75<FC HI<1.25. The distribution of responder/non-responder/unknown classes is shown in FIG. 3.

Conclusion

Three classes of responses to the Starve-Refeed protocol were observed: Responders, non-Responders, and Unknown.

### Example 3

Purpose

This example describes the use of the response to the starve-refeed protocol as a prognostic to assess likelihood of IUI/IVF success.

Materials/Method

Subjects are adult females (e.g., between 18 and 35 years old) without history of recurrent pregnancy loss and may or may not having previously attempted IUI or IVF.

Semen of the male partner is collected prior to IUI insemination or IVF and subjected to Starve-Refeed treatment and responder/nonresponder analysis outlined in Example 2.

Subjects are treated with standard of care medicines (e.g., Clomid preparation, with Hcg triggering injection as indicated) and receive sperm that is either treated by Starve-Refeed as outlined in Example for untreated sperm subjected to IUI standard of care protocol.

Pregnancies are monitored with regular follow-up. Females receiving SR-treated sperm from a partner classified as a responder are expected to exhibit a parameter of improved fertility, for example, increased rate of pregnancy, fetal heart rate (e.g., at 7 weeks), ongoing pregnancy (e.g., at 10 weeks) and/or livebirth rates, relative to females receiving untreated sperm or SR-treated sperm classified as non-responder or unknown.

Conclusion

It is predicted that patients who are responders to starve-refeed as described herein will have a higher rate of success in IUI/IVF procedures relative to patients from the non-responder or unknown classes.

For all patents, applications, or other reference cited herein, such as non-patent literature and reference sequence information, it should be understood that they are incorporated by reference in their entirety for all purposes as well as for the proposition that is recited. Where any conflict exists between a document incorporated by reference and the present application, this application will control. All information associated with reference gene sequences disclosed in this application, such as Gene IDs or accession numbers (typically referencing NCBI accession numbers), including, for example, genomic loci, genomic sequences, functional annotations, allelic variants, and reference mRNA (including, e.g., exon boundaries or response elements) and protein sequences (such as conserved domain structures), as well as chemical references (e.g., PubChem compound, PubChem substance, or PubChem Bioassay entries, including the annotations therein, such as structures and assays, et cetera), are hereby incorporated by reference in their entirety.

Headings used in this application are for convenience only and do not affect the interpretation of this application.

Preferred features of each of the aspects provided by the invention (e.g., media, compositions, preparations, and methods) are applicable to all of the other aspects of the invention mutatis mutandis and, without limitation, are exemplified by the dependent claims and also encompass combinations and permutations of individual features (e.g., elements, including numerical ranges and exemplary embodiments) of particular embodiments and aspects of the invention, including the working examples. For example, particular experimental parameters exemplified in the working examples can be adapted for use in the claimed invention piecemeal without departing from the invention. For example, for materials that are disclosed, while specific reference of each of the various individual and collective combinations and permutations of these compounds may not be explicitly disclosed, each is specifically contemplated and described herein. Thus, if a class of elements A, B, and C are disclosed as well as a class of elements D, E, and F and an example of a combination of elements A-D is disclosed, then, even if each is not individually recited, each is individually and collectively contemplated. Thus, in this example, each of the combinations A-E, A-F, B-D, B-E, B—F, C-D, C-E, and C—F are specifically contemplated and should be considered disclosed from disclosure of A, B, and C; D, E, and F; and the example combination A-D. Likewise, any subset or combination of these is also specifically contemplated and disclosed. Thus, for example, the sub-groups of A-E, B-F, and C-E are specifically contemplated and should be considered disclosed from disclosure of A, B, and C; D, E, and F; and the example combination A-D. This concept applies to all aspects of this application, including elements of a composition of matter and steps of method of making or using the compositions.

The forgoing aspects of the invention, as recognized by the person having ordinary skill in the art following the teachings of the specification, can be claimed in any combination or permutation to the extent that they are novel and non-obvious over the prior art—thus, to the extent an element is described in one or more references known to the person having ordinary skill in the art, they may be excluded from the claimed invention by, inter alia, a negative proviso or disclaimer of the feature or combination of features.

What is claimed is:

1. A method of evaluating sperm quality of a sperm of a human donor, the method comprising:
(a) incubating a sample of sperm obtained from the human donor under energy depletion conditions for a time suitable to generate a potentiated sperm in the sample;
(b) contacting the potentiated sperm in the sample from step (a) with an effective amount of a first energy source, and optionally contacting the potentiated sperm with an effective amount of a second energy source; and
(c) determining a sperm function in the sample from step (b) to evaluate sperm quality, wherein an increase in the sperm function relative to a suitable control indicates a higher sperm quality.

2. The method of claim 1, wherein step (b) comprises contacting the potentiated sperm with the first energy source and the second energy source concurrently.

3. The method of claim 1, wherein step (b) comprises contacting the potentiated sperm with the first energy source and the second energy source sequentially.

4. The method of claim 1, wherein the determining sperm function of step (c) comprises determining motility by computer assisted semen analysis (CASA), sperm capacitation by a sperm-zona pellucida binding assay, ability to fertilize an egg by a sperm penetration assay, autophagy, generation of an embryo, or a combination thereof.

5. The method of claim 4, wherein the motility is determined by curvilinear velocity, amplitude of lateral head displacement, percentage of hyperactivated sperm, percentage of intermediate motility sperm, or a combination thereof.

6. The method of claim 5, wherein the determining of step (c) comprises calculating a ratio of percent of hyperactivated sperm and intermediate motility sperm in the sperm sample relative to that in the suitable control.

7. The method of claim 1, wherein the increase in sperm function comprises an increase curvilinear velocity, amplitude of lateral head displacement, percentage of hyperactivated sperm, percentage of intermediate motility sperm, autophagy, or a combination thereof.

8. The method of claim 1, wherein the increase in sperm function comprises generation of an embryo by the sperm in the sample from step (b), wherein the embryo exhibits longer viability, improved implantation, and/or ability to develop to at least a 2-cell developmental stage, blastocyst developmental stage or an offspring relative to an embryo generated by a suitable control.

9. The method of claim 1, wherein the human donor is oligospermic or subfertile.

10. The method of claim 1, wherein the method is performed at an osmolality ranging from 200-280 mOsm/kg.

11. The method of claim 1, wherein the first energy source is a glycolytic energy source, or a gluconeogenesis substrate.

12. The method of claim 1, wherein the second energy source is a glycolytic energy source or a gluconeogenesis substrate, and wherein the second energy source is one not selected as the first energy source.

13. The method of claim 1, which further comprises:
responsive to a determination of higher sperm quality, contacting sperm from the donor with media suitable for a less-invasive reproductive modality or providing the sperm from the donor with access to an egg by a less-invasive reproductive modality; or
responsive to a determination that the sperm have a lower sperm quality, contacting sperm from the donor with media suitable for a more-invasive reproductive modality or providing the sperm from the donor with access to an egg by a more-invasive reproductive modality.

14. The method of claim 1, further comprising incubating a sample of sperm obtained from the human donor under energy depletion conditions for a time suitable to generate a potentiated sperm in the sample and providing the potentiated sperm in the sample from step with an effective amount of the first energy source, and optionally providing an effective amount of the second energy source.

15. A reproduction method, comprising providing a sperm of a mammalian donor with access to an egg by a reproduction modality, wherein a sperm sample from the mammalian donor was previously evaluated by the method of claim 1.

16. A reproduction method, comprising:
(I) providing a sample of sperm that was:
(a) obtained from a mammalian donor and was incubated under energy depletion conditions for a time suitable to generate a potentiated sperm in the sample;
(b) the potentiated sperm from step (a) was contacted with an effective amount of a first energy source, and optionally an effective amount of a second energy source; and
(c) a sperm function in the sample from step (b) was determined, to evaluate sperm quality, wherein an increase in the sperm function relative to a suitable control indicated a higher sperm quality;
(II) wherein the method further comprises:
responsive to a determination of higher sperm quality, contacting sperm from the donor with media suitable for a less-invasive reproductive modality or providing the sperm from the donor with access to an egg by a less-invasive reproductive modality;
responsive to a determination that the sperm have a lower sperm quality, contacting sperm from the donor with media suitable for a more-invasive reproductive modality or providing the sperm from the donor with access to an egg by a more-invasive reproductive modality.

17. The method of claim 16, wherein the less invasive reproduction modality comprises providing the sperm with access to an egg in vivo, to promote in vivo fertilization of the egg, by IUI, ICI, or IVI, or wherein the more invasive reproduction modality comprises providing the sperm with access to an egg in vitro, to thereby promote in vitro fertilization of the egg, wherein the providing access in vitro comprises incubating the sperm of the mammalian donor with the egg or injecting the sperm of the mammalian donor into the cytoplasm of the egg.

18. The method of claim 16, wherein the reproduction modality comprises incubating a sample of sperm obtained from the mammalian donor under energy depletion conditions for a time suitable to generate a potentiated sperm in the sample and providing the potentiated sperm in the sample from step with an effective amount of the first energy source, and optionally providing an effective amount of the second energy source.

19. A reproduction method, comprising:
(I) providing a sample of sperm that was:
(a) obtained from a mammalian donor and was incubated under energy depletion conditions for a time suitable to generate a potentiated sperm in the sample;
(b) the potentiated sperm from step (a) was contacted with an effective amount of a first energy source, and optionally an effective amount of a second energy source; and
(c) a sperm function in the sample from step (b) was determined, to evaluate sperm quality, wherein an increase in the sperm function relative to a suitable control indicated a higher sperm quality;
(II) wherein the method further comprises:
responsive to a determination of higher sperm quality, incubating a sample of sperm from the donor under energy depletion conditions for a time suitable to generate a potentiated sperm in the sample, and optionally providing an effective amount of the first energy source, and optionally providing an effective amount of the second energy source;
responsive to a determination that the sperm have a lower sperm quality, incubating a sample of sperm from the donor under standard capacitation conditions.

20. The method of claim 19, further comprising providing the sperm of step (II) with access to an egg by a reproductive modality.
